(19) United States
(12) Patent Application Publication
(10) Pub. No.: US 2026/0114948 A1
(43) Pub. Date: Apr. 30, 2026

(54) DRAPE FOR USE IN SURGICAL PROCEDURES

(71) Applicant: Mizuho Orthopedic Systems, Inc., Union CIty, CA (US)

(72) Inventors: Stephan Schmid, San Francisco, CA (US); Vincent Hodges, San Jose, CA (US); Jean-Francois DeBarros, San Francisco, CA (US); Nicholas Matta, San Mateo, CA (US)

(21) Appl. No.: 19/370,426

(22) Filed: Oct. 27, 2025

Related U.S. Application Data
(60) Provisional application No. 63/712,500, filed on Oct. 27, 2024.

(51) Int. Cl. A61B 46/10; A61B 46/00; A61G 13/00; A61G 13/12

(52) U.S. Cl. CPC A61B 46/10; A61B 46/40; A61G 13/0063; A61G 13/0081; A61G 13/125

(57) ABSTRACT

A drape is provided. The drape may support positioning a patient for a surgical procedure and may include one or more hard points. The hard points may be coupled to a medical tool mount disposed in a working space and may releasably grip a medical tool in a sterile space to form a sterile barrier between the medical tool mount and the medical tool.

### RELATED APPLICATION DATA

This application claims priority to U.S. Provisional Application No. 63/712,500, filed Oct. 27, 2024, the disclosure of which is hereby incorporated by reference in its entirety.

### FIELD

This invention relates to medical procedures, and more particularly, to surgical drapes used to reduce contamination of surgical sites.

### BACKGROUND

A surgical procedure is naturally invasive, requiring one or more incisions in a patient to allow a surgeon access to a surgical site. Contamination of the surgical site is a concern in surgical procedures, as infection may lead to consequences, including difficult treatment and mortality. To prevent contamination of the surgical site, healthcare professionals employ various infection control practices, such as ventilation of operating rooms, disinfecting surgical equipment, and maintaining proper patient positioning during a surgical procedure.

Surgical drapes are additional sterilization vehicles that may be used to create a sterile space in which the patient may be positioned. A surgical drape may create a sterile barrier that isolates the surgical site from sources of contamination. These sources may include surfaces of the patient's body, for example, which may host bacteria that are harmful if they enter the patient through an incision.

The sterile barrier may also function to separate the sterile space from other aspects of an operating room that could cause contamination of the surgical site. These aspects may include equipment for properly positioning a patient during the surgical procedure. For example, in a hip replacement procedure, proper positioning of the femur is facilitated by particular equipment. The equipment, while sanitized prior to the surgical procedure, may not be completely free of contaminants. Thus, creating the sterile barrier between the surgical site and the equipment prevents contaminants from a working space containing the equipment from reaching the sterile space. In addition, the sterile barrier may prevent debris from the surgical procedure from reaching the equipment in the working space, which assists healthcare professionals in maintaining the cleanliness of the equipment across multiple surgical procedures.

### SUMMARY

In one general aspect, surgical drape may include a drape top surface configured to contact a sterile space for a surgical procedure. Surgical drape may also include a drape bottom surface opposing the drape top surface and configured to contact a working space, the drape bottom surface and the drape top surface extending between a proximal end and a distal end of the surgical drape. Drape may furthermore include a hard point integral to at least the drape top surface and having a hard point top surface configured to contact the sterile space and a hard point bottom surface configured to contact the working space, the hard point may include a flexible portion having a flexible material configured to releasably grip a medical tool having a connector on at least generally opposing sides of the connector to form a sterile barrier between a medical tool mount disposed in the working space and the medical tool coupled to the medical tool mount in the sterile space. Drape may in addition include where the flexible portion is configured to move in conjunction with the medical tool mount. Other embodiments of this aspect include corresponding computer systems, apparatus, and computer programs recorded on one or more computer storage devices, each configured to perform the actions of the methods.

Implementations may include one or more of the following features.

Surgical drape where the flexible material may include a flexible material shape, and where the medical tool mount may include a claw having a claw shape, the flexible material shape corresponding to the claw shape. Surgical drape where the hard point further may include a recessed portion configured to receive the connector of the medical tool, the connector having a head portion having a first diameter and a shaft having a second diameter, the first diameter larger than the second diameter. Surgical drape where the recessed portion may include a recessed portion shape, and where the head portion may include a head portion shape, the recessed portion shape corresponding to the head portion shape. Surgical drape where the hard point may include a frame surrounding the recessed portion configured to receive the shaft, the frame having a frame opening having a frame diameter corresponding to the second diameter such that the frame opening is configured to receive the shaft but not the head portion having the first diameter, such that the head portion may be retained in the recessed portion by the frame when the hard point is coupled to the medical tool mount. Surgical drape where the recessed portion and the frame may include the flexible material such that the recessed portion and the frame are configured to cinch the connector. Surgical drape where the frame opening may include a v-shaped edge. Surgical drape where the frame opening may include a frame opening shape, and where the head portion may include a head portion shape, the frame opening shape being distinct from the head portion shape. Surgical drape where the frame opening may include a frame opening shape, and where the shaft may include a shaft shape, the frame opening shape corresponding to the shaft shape. Surgical drape where the hard point may include an outer frame surrounding the recessed portion and the frame. Surgical drape where the frame extends from the outer frame. System where the medical tool is a foot mount. System where the hard point may include a first end and a second end configured to move toward the first end to couple the medical tool to the medical tool mount. System where the hard point is configured to incrementally move relative to the medical tool. System where the hard point is more rigid than the drape material. System where the medical tool may include a connector, and where the hard point is less rigid than the connector. System where the medical tool mount is a manipulator arm. Surgical drape where the recessed portion extends below the frame. Surgical drape where the outer frame is oblique relative to a front surface of the frame. Surgical drape where a rear surface of the recessed portion is parallel to the front surface of the frame. System where the second end is configured to incrementally move toward the first end to adjust a grip of the coupling of the medical tool to the medical tool mount. Surgical drape may include: a drape material may include by the top surface and the bottom surface, where the hard point is more rigid than the drape material. System where the hard point may include a gripping portion configured to grip the medical tool on two or more sides, the gripping portion configured to move relative to the medical tool to incrementally grip the medical tool. Surgical drape where the hard point may include a first end, and a second end configured to move toward the first end to couple the medical tool to the medical tool mount. Surgical drape where the hard point may include a channel extending between the first end and the second end, the channel configured to receive at least a portion of the connector, the connector having an elongated member. Surgical drape where the hard point may include a bottom surface, the first end extending above the second end relative to the bottom surface. Surgical drape where the hard point may include a receiving cavity configured to receive the connector of the medical tool. Surgical drape where the hard point may include a gripping portion configured to grip the connector of the medical tool on the at least generally opposing sides, the gripping portion configured to incrementally move relative to the drape material to incrementally grip the medical tool. Surgical drape where the gripping portion may include a first end, and a second end configured to incrementally move toward the first end to incrementally grip the medical tool. Surgical drape where the gripping portion may include a first end, and a second end extending from a bottom surface of the hard point, the first end extending above the second end relative to the bottom surface. Surgical drape where the second end is configured to incrementally move toward the first end to incrementally grip the medical tool. Implementations of the described techniques may include hardware, a method or process, or a computer tangible medium.

Features described above in respect of the preceding aspect may also apply to the next aspect.

In one general aspect, system may include a drape having a drape top surface configured to contact a sterile space for the surgical procedure and a drape bottom surface opposing the drape top surface and configured to contact a working space, the drape bottom surface and the drape top surface extending between a proximal end and a distal end of the drape. System may also include a drape material may include by the drape top surface and the drape bottom surface. System may furthermore include a medical tool mount disposed in the working space, the medical tool mount having a medical tool mount shape. System may in addition include a medical tool in the sterile space coupled to the medical tool mount. System may moreover include a hard point integral to at least the drape top side and having a hard point top surface configured to contact the sterile space and a hard point bottom surface configured to contact the working space, the hard point disposed between the medical tool mount and the medical tool, the hard point may include a flexible portion having a flexible material configured to releasably grip the medical tool on at least generally opposing sides of the medical tool to form a sterile barrier between the medical tool mount disposed in the working space and the medical tool in the sterile space coupled to the medical tool mount. System may also include where the flexible portion may include a flexible portion shape that corresponds to the medical tool mount shape. System may furthermore include where the flexible portion is configured to move in conjunction with the medical tool mount. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

In one general aspect, method may include positioning a drape such that the drape contacts a working space and a sterile space for the surgical procedure, the drape having a drape hard point. Method may also include positioning the drape hard point of the drape against a medical tool mount disposed in the working space. Method may furthermore include releasably gripping a medical tool disposed in the sterile space via the drape hard point such that the medical tool and the medical tool mount are coupled when the medical tool is gripped. Other embodiments of this aspect include corresponding computer systems, apparatus, and computer programs recorded on one or more computer storage devices, each configured to perform the actions of the methods.

Implementations may include one or more of the following features.

Method where releasably gripping the medical tool disposed in the sterile space via the drape hard point may include adjusting the drape hard point such that the drape hard point may include a drape hard point shape that corresponds with a medical tool shape of the medical tool. Method where releasably gripping the medical tool disposed in the sterile space via the drape hard point may include gripping the medical tool on the at least generally opposing sides. Method where releasably gripping the medical tool disposed in the sterile space via the drape hard point may include incrementally gripping the medical tool. Method where releasably gripping the medical tool disposed in the sterile space via the drape hard point may include moving first end of the drape hard point toward a second end of the drape hard point. Method where the first end is incrementally moved toward the second end. Method where positioning the drape hard point against the medical tool mount may include corresponding a flexible portion shape of a flexible portion of the drape hard point with a medical tool mount shape of the medical tool mount, the flexible portion having a flexible material. Method where releasably gripping the medical tool disposed in the sterile space via the drape hard point may include moving the flexible portion in conjunction with the medical tool mount. Method where the medical tool is a foot mount. Method where the medical tool mount is a manipulator arm. Implementations of the described techniques may include hardware, a method or process, or a computer tangible medium.

In one general aspect, manipulator arm surgical drape may include an arm drape. Manipulator arm surgical drape may also include a table drape coupled to the arm drape. Drape may furthermore include a hard point integral to the arm drape, the hard point configured to contact a sterile space for a surgical procedure and a working space having a medical tool mount having a manipulator arm configured to receive the arm drape, the hard point configured to contact the sterile space and the working space, and the hard point having a flexible portion having a flexible material configured to releasably grip the medical tool mount. Drape may in addition include where the flexible portion is configured to move in conjunction with the medical tool mount. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

Implementations may include one or more of the following features.

Surgical drape may include: a pouch extending from the arm drape, the pouch extending perpendicularly from the arm drape. Surgical drape where the arm drape further may include one or more labels configured to indicate a step for unpackaging the manipulator arm surgical drape. Surgical drape where the arm drape may include one or more films, each of the one or more films having at least one of the one or more labels. Surgical drape where the one or more labels may include a tear here label. Surgical drape where the one or more labels may include an apply label. Surgical drape where the one or more labels may include an apply here label. Surgical drape where the one or more labels may include a table orientation label. Surgical drape where the one or more labels may include an insert hand label. Surgical drape where the one or more labels may include a peel here label. Surgical drape where the arm drape and the table drape each may include different materials. Surgical drape where the table drape may include a fabric material. Implementations of the described techniques may include hardware, a method or process, or a computer tangible medium.

In one general aspect, method may include folding the manipulator arm surgical drape, the manipulator arm surgical drape having: an arm drape; a table drape coupled to the arm drape; and a hard point integral to the arm drape, the hard point configured to contact a sterile space for a surgical procedure and a working space having a medical tool mount having a manipulator arm configured to receive the arm drape, the hard point configured to contact the sterile space and the working space, and the hard point having a flexible portion having a flexible material configured to releasably grip the medical tool mount, where the flexible portion is configured to move in conjunction with the medical tool mount. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

Implementations may include one or more of the following features.

Method where folding the manipulator arm surgical drape may include: accordion folding at least a portion of the table drape in a first fold; folding at least a portion of the arm drape over the first fold in a second fold; folding sides of the table drape in a fourth fold; adding one or more labels to the table drape; and folding the arm drape such that the hard point faces toward the manipulator arm surgical drape. Implementations of the described techniques may include hardware, a method or process, or a computer tangible medium.

Features described above in respect of any of the preceding aspects may also apply to the next aspect.

Implementations may include one or more of the following features.

Aspects provide a system. The system may include a medical tool mount disposed in a working space for a surgical procedure and including a medical tool mount shape. The system may also include a medical tool disposed in a sterile space for the surgical procedure and coupled to the medical tool mount, the medical tool including a connector including generally opposing sides. The system may also include a drape configured to separate the sterile space from the working space. The drape may include a proximal end, a distal end, a drape top surface configured to extend between the proximal end and the distal end of the drape and further configured to define the boundary of the sterile space, a drape bottom surface opposing the drape top surface, the drape bottom surface configured to extend between the proximal end and the distal end of the drape and further configured to define the boundary of the working space, and a hard point integral to at least the drape top surface and disposed between the medical tool mount and the medical tool. The hard point may include a hard point top surface configured to contact the sterile space, a hard point bottom surface configured to contact the working space, and a flexible portion including a flexible material configured to releasably grip the connector of the medical tool on at least the generally opposing sides of the connector. The hard point may be configured to form a sterile barrier between the medical tool mount disposed in the working space and the medical tool disposed in the sterile space and coupled to the medical tool mount. The flexible portion may be configured to move in conjunction with the medical tool mount as it releasably grips at least the generally opposing sides of the connector of the medical tool, such that the hard point maintains the sterile barrier before and after the flexible portion releasably grips at least the generally opposing sides of the connector.

In some aspects, the flexible material includes a flexible material shape. The medical tool mount includes a claw having a claw shape, the flexible material shape including a corresponding claw shape.

In some aspects, the hard point further includes a recessed pocket configured to receive at least a portion of the connector of the medical tool, the connector including a head portion including a first diameter and a shaft including a second diameter, the first diameter larger than the second diameter.

In some aspects, the recessed pocket includes a bottom shape. The head portion includes a head portion shape, the bottom shape corresponding to the head portion shape.

In some aspects, the medical tool is a foot mount.

In some aspects, the flexible portion of the hard point includes a first end and a second end configured to move generally toward the first end to releasably grip the connector of the medical tool on at least the generally opposing sides of the connector.

In some aspects, the first end is configured to move toward the second end.

In some aspects, the drape further includes a drape material, and the hard point is more rigid than the drape material.

In some aspects, the hard point is less rigid than the connector.

Features described above in respect of any of the preceding aspects may also apply to the next aspect.

Aspects provide a system. The system may include a medical tool mount disposed in a working space for a surgical procedure and including a medical tool mount shape. The system may also include a medical tool disposed in a sterile space for the surgical procedure and coupled to the medical tool mount. The medical tool may include a connector including generally opposing sides. The system may also include a drape configured to separate the sterile space from the working space. The drape may include a proximal end, a distal end, a drape top surface configured to extend between the proximal end and the distal end of the drape and further configured to define the boundary of the sterile space, a drape bottom surface opposing the drape top surface, the drape bottom surface configured to extend between the proximal end and the distal end of the drape and further configured to define the boundary of the working space, and a hard point integral to at least the drape top side and disposed between the medical tool mount and the medical tool. The hard point may include a hard point top surface configured to contact the sterile space, a hard point bottom surface configured to contact the working space, a recessed pocket configured to receive the head of the connector, a frame surrounding the recessed pocket and configured to receive the shaft of the connector, and a flexible portion including a flexible material configured to releasably grip the connector of the medical tool on at least the generally opposing sides of the connector. The hard point may be configured to form a sterile barrier between the medical tool mount disposed in the working space and the medical tool disposed in the sterile space and coupled to the medical tool mount.

In some aspects, the flexible portion is configured to move in conjunction with the medical tool mount as it releasably grips at least the generally opposing sides of the connector of the medical tool, such that the hard point maintains the sterile space and the working space before and after the flexible portion releasably grips at least the generally opposing sides of the connector. The recessed pocket and the frame include the flexible material such that the recessed pocket and the frame are configured to cinch at least the generally opposing sides of the connector.

In some aspects, the hard point further includes a rear surface, a recessed cavity formed in the recessed pocket, and an outer frame surrounding the recessed pocket and the frame. The frame includes transverse walls extending generally transversely from the rear surface, a front surface generally parallel to the rear surface, a frame opening formed in the front surface and including a v-shaped edge, and a frame cavity bounded by the rear surface, the transverse walls, and the front surface. The recessed cavity extends below the frame cavity. The outer frame is circular and oblique relative to the front surface of the frame. The recessed pocket extends outwardly from and below the outer frame. The connector further includes a head portion including a first diameter and a shaft including a second diameter, the first diameter larger than the second diameter. The recessed pocket includes a bottom shape, the recessed cavity includes a recessed cavity shape, and the head portion includes a head portion shape, the recessed cavity shape corresponding to the bottom shape and the head portion shape, the recessed cavity shape, the bottom shape, and the head portion shape being octagonal. The frame opening has a frame diameter corresponding to the second diameter such that the frame opening is configured to receive the shaft but not the head portion including the first diameter, such that the head portion may be retained in the recessed cavity by the frame when the hard point is coupled to the medical tool mount.

In some aspects, the frame includes a frame opening including a v-shaped edge. The frame opening includes a frame opening shape. The shaft includes a shaft shape, the frame opening shape corresponding to the shaft shape.

In some aspects, the hard point further includes an outer frame surrounding the recessed pocket and the frame. The frame includes a front surface. The outer frame is oblique relative to the front surface of the frame.

Features described above in respect of any of the preceding aspects may also apply to the next aspect.

Aspects provide a system. The system may include a medical tool mount disposed in a working space for a surgical procedure and including a medical tool mount shape. The system may also include a medical tool disposed in a sterile space for the surgical procedure and coupled to the medical tool mount. The medical tool may include a connector including generally opposing sides. The system may also include a drape configured to separate the sterile space from the working space. The drape may include a proximal end, a distal end, a drape top surface configured to extend between the proximal end and the distal end of the drape and further configured to define the boundary of the sterile space, a drape bottom surface opposing the drape top surface, the drape bottom surface configured to extend between the proximal end and the distal end of the drape and further configured to define the boundary of the working space, and a hard point integral to at least the drape top side and disposed between the medical tool mount and the medical tool. The hard point may include a hard point top surface configured to contact the sterile space, a hard point bottom surface configured to contact the working space, a first end, a second end, and, a flexible portion including a flexible material configured to releasably grip the connector of the medical tool on at least the generally opposing sides of the connector. The hard point may be configured to form a sterile barrier between the medical tool mount disposed in the working space and the medical tool disposed in the sterile space and coupled to the medical tool mount. The flexible portion may be configured to move in conjunction with the medical tool mount as it releasably grips at least the generally opposing sides of the connector of the medical tool, such that the hard point maintains the sterile space and the working space before and after the flexible portion releasably grips at least the generally opposing sides of the connector. The second end may be configured to move toward the first end to couple the medical tool to the medical tool mount.

In some aspects, the hard point further includes a channel extending between the first end and the second end, the channel configured to receive at least a portion of the connector and a bottom surface of the channel, the first end extending above the second end relative to the bottom surface of the channel. The bottom surface of the channel is oblique relative to the hard point bottom surface. The flexible material includes a flexible material shape. The medical tool mount includes a claw having a claw shape, the flexible material shape corresponding to the claw shape such that the second end is configured to move toward the first end to couple the medical tool to the medical tool mount.

In some aspects, the first end is configured to move toward the second end to couple the medical tool to the medical tool mount.

In some aspects, the flexible material includes a flexible material shape. The medical tool mount includes a claw having a claw shape, the flexible material shape corresponding to the claw shape.

In some aspects, The hard point further includes a receiving opening configured to receive at least a portion of the connector of the medical tool.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 shows a perspective view of a hip drape according to an exemplary embodiment.

FIG. 2 shows a perspective view of a medical tool according to an exemplary embodiment.

FIG. 3 shows an enlarged perspective view of the hip drape of FIG. 1.

FIG. 4 shows an enlarged perspective view of the hip drape of FIG. 1.

FIG. 5 shows an enlarged top view of the hip drape of FIG. 1.

FIG. 6 shows a schematic of the hip drape of FIG. 1.

FIG. 7 shows a perspective view of a medical tool mount according to an exemplary embodiment.

FIG. 8 shows a schematic of a portion of the hip drape of FIG. 1 and the medical tool mount of FIG. 7.

FIG. 9 shows a schematic of a portion of the hip drape of FIG. 1 and the medical tool mount of FIG. 7.

FIG. 10 shows a schematic of a portion of the hip drape of FIG. 1, the medical tool of FIG. 2, and the medical tool mount of FIG. 7.

FIG. 11 shows a perspective view of a portion of the hip drape of FIG. 1, the medical tool of FIG. 2, and the medical tool mount of FIG. 7.

FIG. 12 shows a perspective view of a hard point and a medical tool mount according to an exemplary embodiment.

FIG. 13 shows a perspective view of the medical tool mount of FIG. 12.

FIG. 14 shows a perspective view of a portion of the medical tool mount of FIG. 12.

FIG. 15 shows a front view of the hard point of FIG. 12

FIG. 16 shows a perspective view of the hard point of FIG. 12.

FIG. 17 shows a cross-section view of the hard point of FIG. 16 along line 17-17.

FIG. 18 shows a perspective view of the hard point of FIG. 12.

FIG. 19 shows a side view of the hard point of FIG. 12.

FIG. 20 shows a cross-section view of the hard point and the medical tool mount of FIG. 12 along line 20-20.

FIG. 21 shows a perspective view of a knee drape according to an exemplary embodiment.

FIG. 22 shows a perspective view of a medical tool mount according to an exemplary embodiment.

FIG. 23 shows a perspective view of a medical tool according to an exemplary embodiment.

FIG. 24 shows a perspective view of the medical tool mount of FIG. 22 and the medical tool of FIG. 23.

FIG. 25 shows an enlarged perspective view of the knee drape of FIG. 21.

FIG. 26 shows an enlarged perspective view of the knee drape of FIG. 21.

FIG. 27 shows an enlarged perspective view of the knee drape of FIG. 21.

FIG. 28 shows a perspective view of an arm drape according to an exemplary embodiment.

FIG. 29 shows a rear view of the arm drape of FIG. 28.

FIG. 30 shows a plan view of the arm drape of FIG. 28.

FIG. 31 shows a front view of the arm drape of FIG. 28.

FIG. 32 shows a label for one or more drapes according to an exemplary embodiment.

FIG. 33 shows a label for one or more drapes according to an exemplary embodiment.

FIG. 34 shows a label for one or more drapes according to an exemplary embodiment.

FIG. 35 shows a label for one or more drapes according to an exemplary embodiment.

FIG. 36 shows a label for one or more drapes according to an exemplary embodiment.

FIG. 37 shows a label for one or more drapes according to an exemplary embodiment.

FIG. 38 shows a folded configuration of the arm drape of FIG. 28.

FIG. 39 shows a folded configuration of the arm drape of FIG. 28.

FIG. 40 shows a folded configuration of the arm drape of FIG. 28.

FIG. 41 shows a folded configuration of the arm drape of FIG. 28.

FIG. 42 shows a folded configuration of the arm drape of FIG. 28.

FIG. 43 shows a folded configuration of the arm drape of FIG. 28.

FIG. 44 shows a folded configuration of the arm drape of FIG. 28.

FIG. 45 shows a folded configuration of the arm drape of FIG. 28.

FIG. 46 shows a folded configuration of the arm drape of FIG. 28.

FIG. 47 shows a folded configuration of the arm drape of FIG. 28.

FIG. 48 shows a folded configuration of the arm drape of FIG. 28.

FIG. 49 shows a folded configuration of the arm drape of FIG. 28.

FIG. 50 shows a folded configuration of the arm drape of FIG. 28.

FIG. 51 shows a folded configuration of the arm drape of FIG. 28.

FIG. 52 shows a perspective view of a lower drape according to an exemplary embodiment.

FIG. 53 shows a perspective view of a lower drape according to an exemplary embodiment.

FIG. 54 shows a perspective view of the medical tool mount of FIG. 12.

### DETAILED DESCRIPTION

U.S. Patent Application No. ______ filed Oct. 27, 2025 titled “SYSTEMS AND METHODS FOR SURGICAL POSITIONING”, U.S. Patent Application No. ______ Filed Oct. 27, 2025 titled SYSTEMS AND METHODS FOR SURGICAL POSITIONING, U.S. Patent Application No. ______ Filed October 2027 titled ARM SUPPORT FOR SURGICAL POSITIONING, U.S. Patent Application No. ______ Filed Oct. 27, 2025 titled SYSTEMS AND METHODS FOR SURGICAL POSITIONING, and U.S. Patent Application No. ______ Filed Oct. 27, 2025 titled SYSTEMS AND METHODS FOR SURGICAL POSITIONING are hereby incorporated by reference in their entirety.

In the following detailed description of embodiments, reference is made to the accompanying drawings, which form a part hereof and in which are shown, by way of illustration, specific embodiments in which the invention may be practiced. Specific details disclosed herein are in every case a non-limiting embodiment representing concrete ways in which the concepts of the invention may be practiced. This serves to teach one skilled in the art to employ the present invention in virtually any appropriately detailed system, structure or manner consistent with those concepts. It will be seen that various changes and alternatives to the specific described embodiments and the details of those embodiments may be made within the scope of the invention. Because many varying and different embodiments may be made within the scope of the inventive concepts herein described and in the specific embodiments herein detailed without departing from the scope of the present invention, it is to be understood that the details herein are to be interpreted as illustrative and not as limiting.

A surgical drape is a tool for defining and maintaining the sterile space of a surgical procedure, and particularly, maintaining the sterility of the surgical site on the patient comprising the incision. The surgical drape may cover the patient, including the surgical site. In certain embodiments, at the surgical site, the surgical drape may have an opening, or a fenestration, to allow access to the patient for the incision work. Across the surgical site may be a film that adheres the drape to the patient such that contaminants originating from the patient's body do not enter the surgical site. In certain embodiments, the area above the drape is generally considered to be sterile, and may be referred to herein as the “sterile space, while the area below the drape is generally considered to be non-sterile, and may be referred to herein as the “working space.” In this way, the surgical drape defines the sterile space as the area above the drape, as well as the working space as the area below the drape. The surgical drape can be considered a sterile barrier between the sterile space and the working space.

Surgeries may be lengthy and may require maintaining a sterile space throughout the duration of procedures. Some procedures may require equipment, such as patient positioning apparatuses to additionally maintain the position of a patient. In certain embodiments, the equipment may be reusable for multiple different surgeries for different patients. For example, hip replacement procedures may utilize equipment for proper positioning of the femur. In knee replacement procedures, equipment may support proper positioning of the operative leg. A surgical drape, in addition to covering the patient, may cover this equipment. The equipment, separated from the sterile space by the surgical drape, may be included in the working space. The surgical drape may prevent debris from surgeries from reaching the equipment in order to maintain the cleanliness of the equipment. To both aid in separating the working space from the sterile space and limit disturbance of the sterile space such that surgical site may remain sterile and the surgical drape surrounding the site may be maintained, it may be important to secure the surgical drape to various points of the working space. In this way, unintended movement of the surgical drape may be prevented, which could cause breach of the sterile barrier. Further, the additional points of coupling may reduce pressure on the adherence of the drape around the surgical site.

Systems and methods described herein describe embodiments of a surgical drape. The surgical drape may contact a sterile space for a surgical procedure and a working space in which equipment may be disposed. One or more hard points integral to the drape may secure the drape to the equipment. The surgical drape having the hard points may create a sterile barrier between the sterile space and the working space. Reference will now be made to the figures.

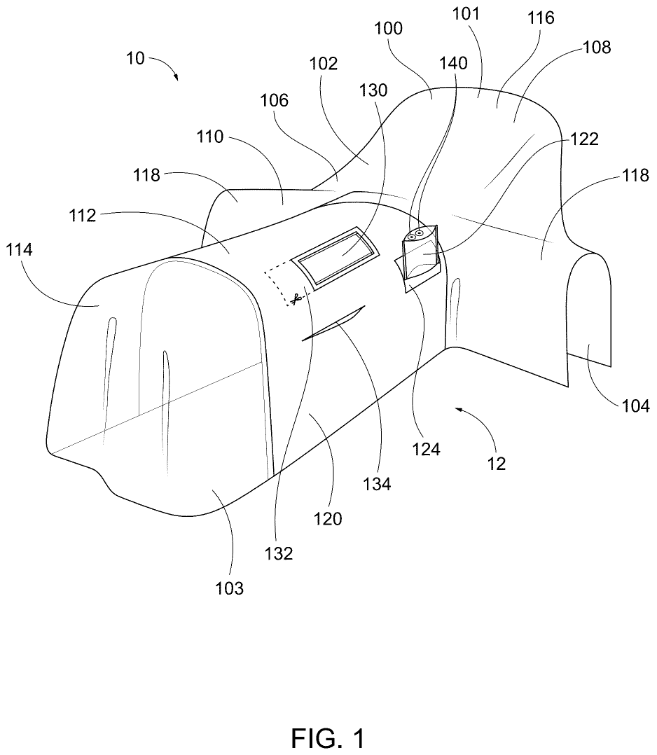

FIG. 1 shows a perspective view of a drape 100 according to an exemplary embodiment. Drape 100 may cover a patient and various equipment, such as an operating table and related accessories, during a surgical procedure to support positioning a patient for a surgical procedure. As shown, drape 100 may contact a sterile space 10, which may include a surgical site. On the other side, drape 100 may contact a working space 12, which may include various equipment for facilitating a surgical procedure. Drape 100 may be a boundary between sterile space 10 and working space 12 such that drape 100 separates sterile space 10 from working space 12. In this way, drape 100 may define sterile space 10 along its top surface 102 and working space 12 along its bottom surface 104.

Working space 12 may include various equipment for facilitating a surgical procedure, and may also include non-operative equipment. Working space 12 may also include portions of the patient that are not the subject of the surgical procedure, e.g., an arm of the patient if the patient is undergoing knee or hip surgery. Working space 12 may also generally include the area below a waist of a practitioner. Equipment and portions of the patient from sterile space 10 that are lowered below the waist of the practitioner during the course of a surgical procedure may be considered non-sterile.

In certain embodiments, a drape material 106 of drape 100 may be separated into one or more sections to cover the patient and various equipment. For example, drape material 106 may be separated into a first section 108, a second section 110, and a third section 112. First section 108 may include a head portion 116 to cover a head of the patient. Second section 110 may include one or more arm portions 118 to cover arms of the patient. Third section 112 may cover an operative leg and/or a non-operative leg, each of which may be supported under drape 100 in working space 12 on various equipment, such as a bed, spar, or well-leg holder. In certain embodiments, first section 108 and second section 110 may include typical drape material, such as composite SMS (Spunbond Meltblown Spunbond) non-woven fabric. First section 108 may include level 3 SMS material, for example. Second section 110 may include level 4 SMS material, such as level 3 SMS material with fluid barrier material. In certain embodiments, drape 100 may also include a liner 114 coupled to third section 112. Liner 114 may include clear polyurethane material. Liner 114 may be clear to facilitate access to and visualization of equipment disposed under drape 100 in working space 12.

In certain embodiments, drape 100 may be structured and configured for particular surgeries, such as hip surgery. Accordingly, drape 100 may include a hip fenestration portion 130, such as along third section 112 of drape 100. In other embodiments, hip fenestration portion 130 may additionally or alternatively be disposed along other sections of drape 100. In other embodiments, other fenestration portion(s) may additionally or alternatively be disposed along other sections of drape 100. Hip fenestration portion 130 may include peel-away tape that may expose an inner adhesive for application to the patient. Hip fenestration portion 130 may be extended for enlargement of the exposure via a hip fenestration portion extension 132. In some embodiments, a fenestration pouch 134 disposed on drape 100, such as along third section 112, may facilitate access to equipment disposed under drape 100 in working space 12.

As shown, drape 100 may include a drape top surface 102 to contact sterile space 10 for a surgical procedure. Drape 100 may also include a drape bottom surface 104 opposing drape top surface 102 to contact working space 12. Drape top surface 102 and drape bottom surface 104 may extend between a proximal end 101 and a distal end 103 of drape 100.

Drape top surface 102 and drape bottom surface 104 may include drape material 106. Drape material 106 may be generally flexible material. In some embodiments, drape 100 may include one or more hard points 140. Hard points 140 may support positioning a patient for a surgical procedure. Additionally or alternatively, hard points 140 may support securing drape 100 to surgical equipment in working space 12, and to secure surgical equipment in sterile space 10 to the surgical equipment in working space 12. In certain embodiments, hard point 140 may be more rigid than drape material 106. In other embodiments, despite being more rigid than drape material 106, hard point 140, and any hard point, may be made of material that is able to be formed, and is also flexible. The forming process may include injection molding, extrusion, thermoforming, rotational molding, and the like. The flexible material may be a elastomeric, for example a thermoplastic, a thermoplastic elastomer, a rubber, a resin, a silicone, and the like. In certain embodiments, hard point 140, and any hard point, may include polyurethane (PU). In some embodiments, the polyurethane may include a durometer range from about 60 A to about 95 A, or about 50 D to about 60 D. In still further embodiments, a hard point includes a material that provides enough rigidity to maintain the sterile barrier under the load and contact between medical tool mounts and medical tools described herein and otherwise, but flexible enough to move with the medical tool mount and securely grip the medical tool. In other embodiments, the material comprising the hard point is compatible with the drape materials such that the hard point and drape can be ultrasonically welded together or bonded with adhesive.

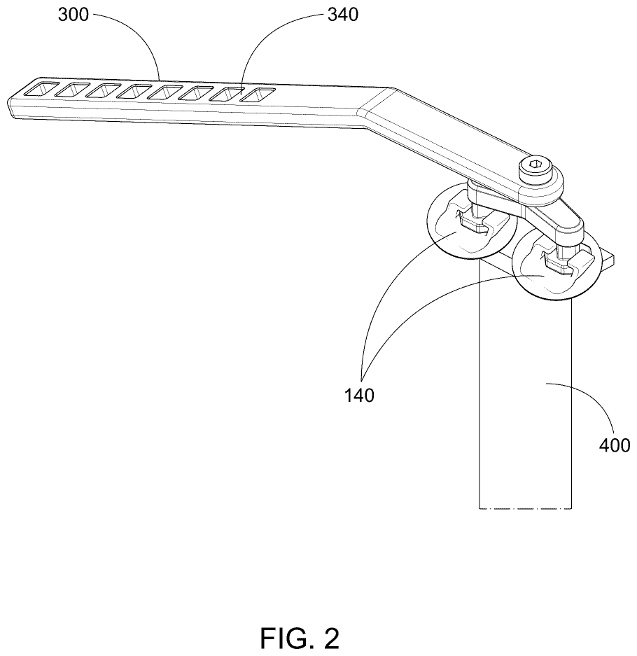

In various embodiments, hard point 140 may be disposed at particular locations on drape 100 to couple drape 100 to other components of the operating room environment, such as equipment in working space 12. For example, one or more hard points 140 may be disposed along third section 112. Hard point 140 may move relative to drape material 106 to grip a medical tool 300 (FIG. 2) and a medical tool mount 400 (FIG. 2) such that hard point 140 may adjust a grip of the coupling of medical tool mount 400 to medical tool 300 during a surgical procedure without tearing drape 100 and breaching the sterile barrier.

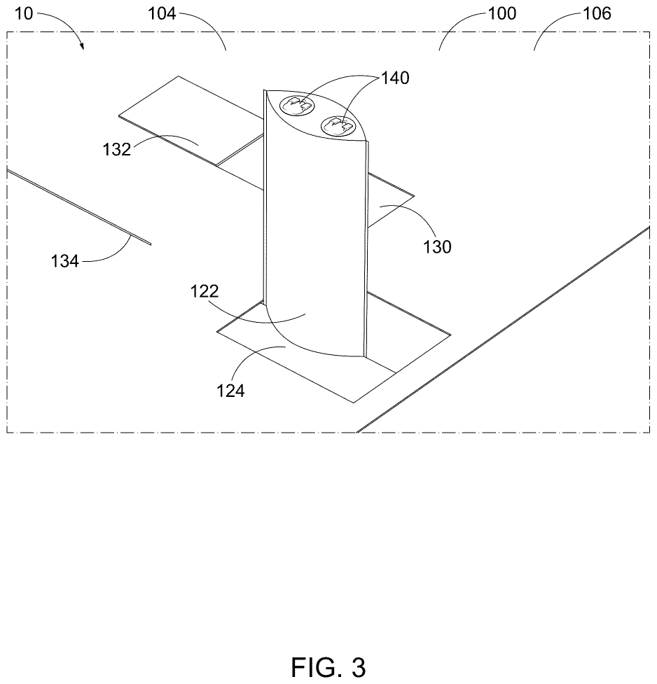

Referring to FIGS. 2-3 according to exemplary embodiments, hard points 140 may couple drape 100 to a medical tool mount 400, shown in FIG. 2. Medical tool mount 400 may be equipment disposed in working space 12 (FIG. 1) for positioning a patient for a surgical procedure and supporting one or more medical tools 300 in sterile space 10 specific to a surgical procedure to position a patient for the surgical procedure. Hard point 140 may be disposed above medical tool mount 400 such that hard point 140 may form a sterile barrier between medical tool mount 400 disposed in working space 12 and sterile space 10. While medical tool mount 400 is disposed in working space 12, medical tool 300, an exemplary embodiment of which is shown in FIG. 2, may be disposed in sterile space 10. Hard point 140 may be disposed between medical tool mount 400 and medical tool 300 to form a sterile barrier between medical tool mount 400 disposed in working space 12 and medical tool 300 coupled to medical tool mount 400 in sterile space 10.

In one embodiment, medical tool 300 shown in FIG. 2 may be a femur hook 340, which may be used in hip surgeries. In other embodiments, medical tool 300 may be a foot mount, e.g., foot mount 3030, for example. Medical tool mount 400 may be structured to support medical tool 300, such as femur hook 340. An example of a medical tool mount 400 is a femur hook arm. Another example is a manipulator arm (not shown).

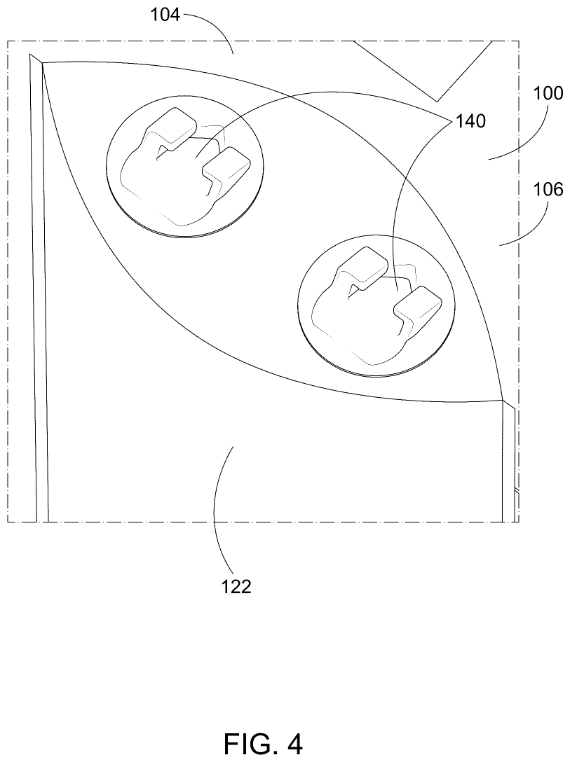

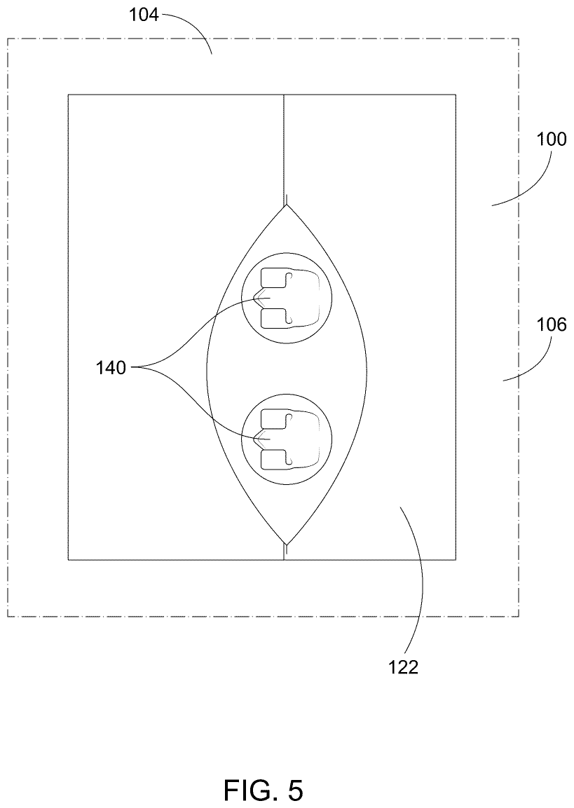

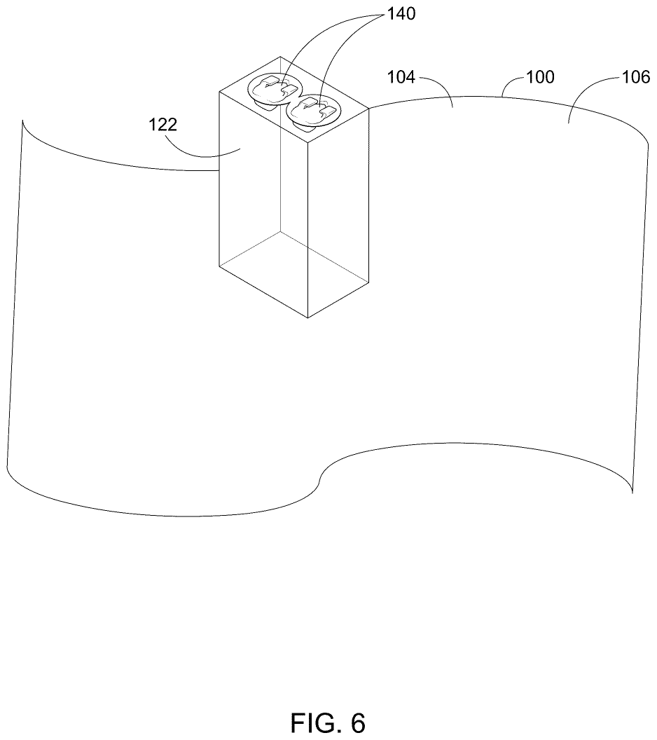

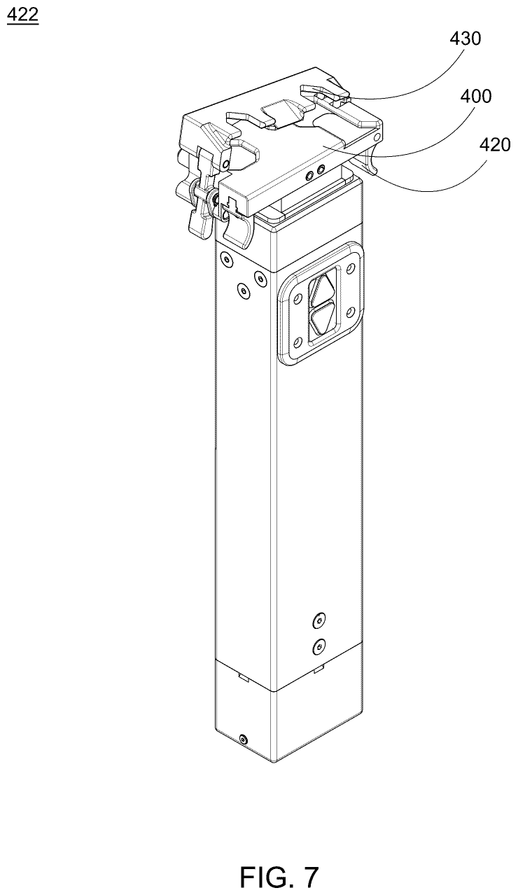

With reference to FIGS. 3-6 according to exemplary embodiments, drape 100 may be shaped to accommodate medical tool mount 400 (FIG. 7). As shown, drape 100 may be generally planar. With reference to FIGS. 3 and 6, drape 100 may form a sleeve 122, or pocket, made of film, or a transparent material, that receives medical tool mount 400. Sleeve 122 may extend from an anchor portion 124 of drape 100. Anchor portion 124 may be a portion of drape 100 from which sleeve 122 protrudes. In some embodiments, anchor portion 124 may be reinforced, or thicker, or rigid, in comparison to surrounding portions of drape 100. In embodiments, drape 100 may include one or more hard points 140, such as about two hard points 140, along sleeve 122 to couple to medical tool mount 400 and medical tool 300. In some embodiments, hard point 140 may be integral to drape 100, such as to at least drape top surface 102. For example, hard point 140 may be integral to drape top surface 102 and drape bottom surface 104 (FIG. 1). In other words, hard point 140 may be formed with drape material 106 or may be joined with drape material 106 such that hard point 140 is coupled to and/or not removable from drape 100. Drape 100 with drape material 106 and one or more hard points 140 combined may create the sterile barrier. Hard points 140 disposed between medical tool 300 (FIG. 2) and medical tool mount 400 (FIG. 2) may form a sterile barrier between medical tool 300 and medical tool mount 400.

In embodiments, hard point 140 may be joined with drape material 106 in any method. For example, hard point 140 may fasten to drape material 106 with adhesive, or it may be sewn. Additionally or alternatively, hard point 140 may be chemically bonded with drape material 106, such as chemically welded, such as with ultrasonic welding or thermal welding.

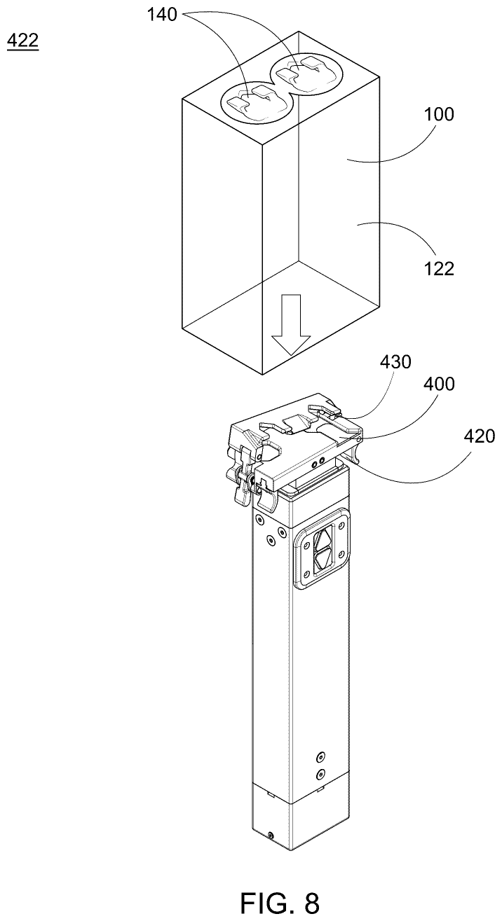

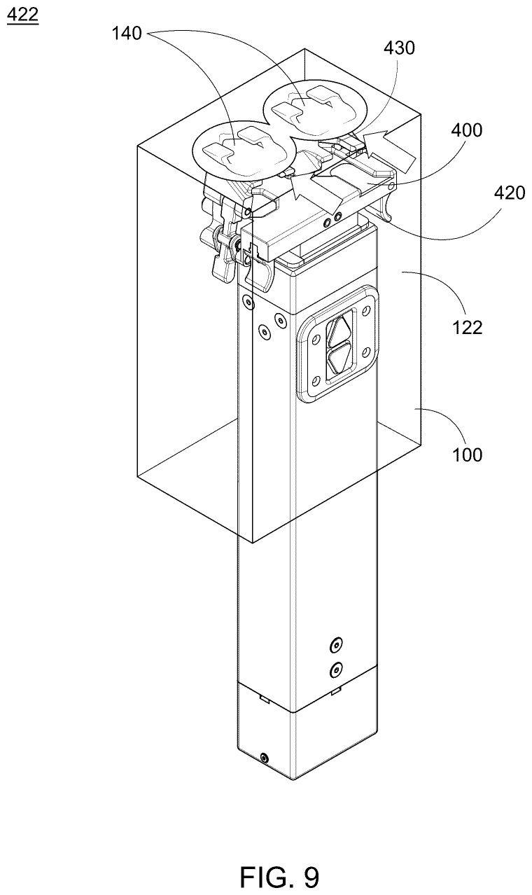

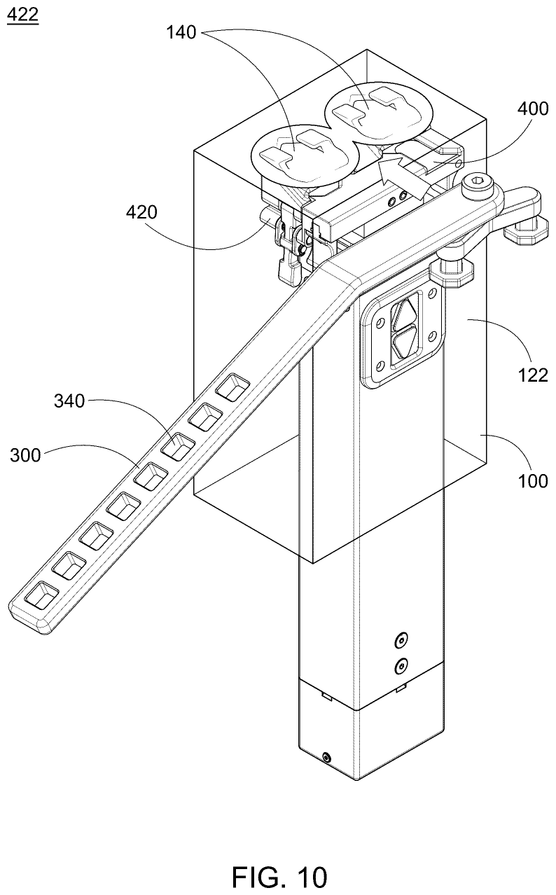

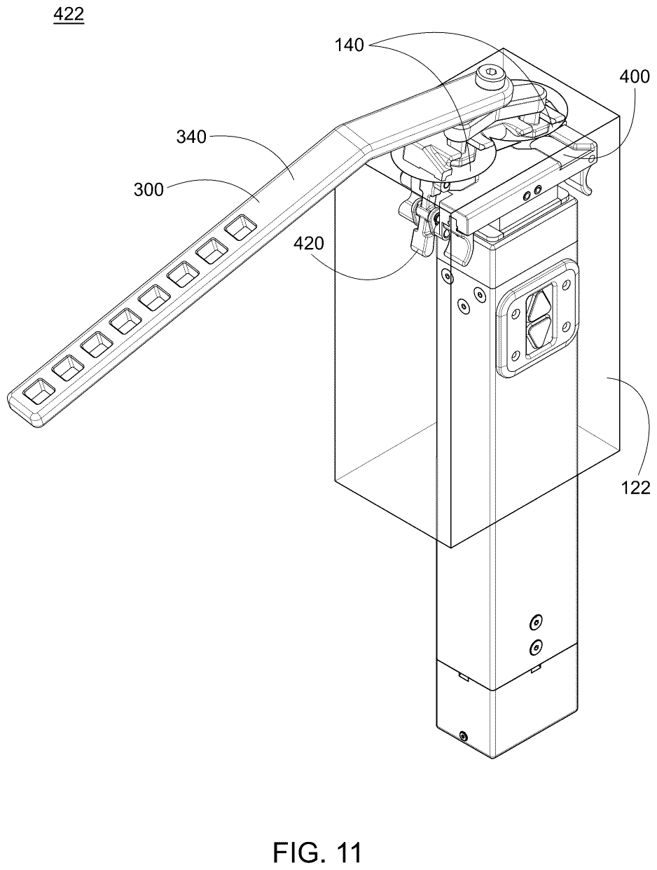

Drape 100 coupled to medical tool mount 400 and medical tool 300 may be seen in FIGS. 7-11 according to exemplary embodiments. As shown, medical tool mount 400 may receive drape 100. For example, sleeve 122 of drape 100 may be disposed around medical tool mount 400 such that sleeve 122 envelopes medical tool mount 400. Medical tool mount 400 may receive one or more hard points 140 along drape 100, such as along sleeve 122.

In embodiments, hard points 140 may include one or more shapes that correspond to the shape of medical tool mount 400. In this way, hard points 140 may mount to medical tool mount 400, and particularly to portions of medical tool mount 400 that may grip medical tool 300. In other embodiments, hard points 140 may include one or more shapes that correspond to the shape of any medical tool mount, such that hard points 140 may mount to such medical tool mount, and particularly to portions of the medical tool mount that may grip a portion of a medical tool. In an example, hard points 140 may fit directly over the shape of medical tool mount 400, or any medical tool mount, such that the shapes of each substantially correspond, like a “hand in glove” arrangement. The clearance between portions of hard point 140 and medical tool mount 400, or any medical tool mount, may be minimal or non-existent. In other words, portions of the hard point 140 may make contact with medical tool mount 400, or any medical tool mount, particularly the portions of a medical tool mount that move in order to grip a medical tool. In addition, hard point 140 may be flexible or resilient such that hard point 140 may move with medical tool mount 400, or portions of medical tool mount 400. Accordingly, as medical tool mount 400 is moved, or portions of medical tool mount 400 are moved, hard point 140 may move without causing separation between hard point 140 and medical tool mount 400, and without bunching or folding of the hard point 140 material.

Hard points 140, while coupled to medical tool mount 400, may releasably grip medical tool 300 to couple medical tool mount 400 and hard point(s) 140 to medical tool 300. While coupled, hard points 140 may be disposed between medical tool mount 400 and medical tool 300 to separate medical tool mount 400 in working space 12 (FIG. 1) and medical tool 300 in sterile space 10 (FIG. 1), thereby maintaining the sterile barrier between medical tool mount 400 in working space 12 and medical tool 300 in sterile space 10.

Hard points 140 may be coupled to medical tool mount 400 and medical tool 300 such that medical tool mount 400 and medical tool 300 are coupled to one another through hard points 140. In other words, medical tool mount 400 may be coupled to medical tool 300 through drape 100, such as through hard points 140. The coupling may be secured as shown such that sterile space 10 (FIG. 1) is maintained for the duration of a surgical procedure. Sterile space 10 may be maintained even while a medical tool 300 is repeatedly coupled and uncoupled from medical tool mount 400.

In certain embodiments, sterile space 10 (FIG. 1) is maintained for the duration of a surgical procedure even if medical tool 300 is decoupled from hard points 140 and medical tool mount 400. In other words, medical tool 300 may be decoupled from hard points 140 without breaching the sterile barrier formed by drape 100. Medical tool 300 may be coupled again to hard point 140 and medical tool mount 400 without breaching the sterile barrier. Hard points 140 may be flexible or resilient such that medical tool 300 may be removed from medical tool mount 400, or may be movable while coupled to medical tool mount 400 through hard point 140, without causing drape 100 to tear. In addition, hard points 140 may be flexible or resilient such that hard point 140 may be removed from medical tool mount 400, or may be movable while coupled to medical tool mount 400, without causing drape 100 to tear. In embodiments, medical tool 300 and medical tool mount 400 may be engaged and disengaged via hard point 140 a plurality of times without compromising or tearing hard point 140 or drape 100 surrounding hard point 140. Hard points 140 may secure both medical tool mount 400 and medical tool 300 such that decoupling is prevented and risk of sterile space 10 being contaminated or non-sterile is mitigated. Drape 100 with hard points 140, therefore, may create the sterile barrier between medical tool mount 400 in working space 12 and medical tool 300 in sterile space 10.

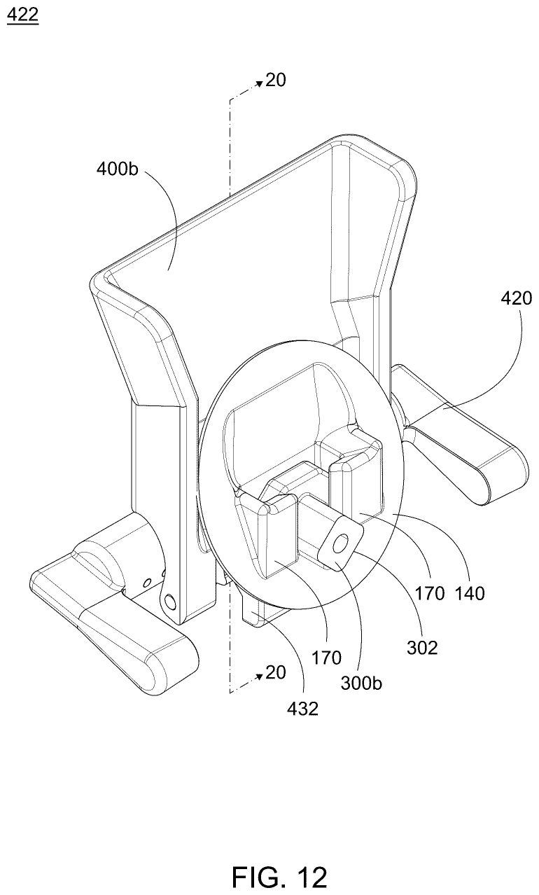

In certain embodiments, hard points 140 may be configured to be compatible with a plurality of medical tool mounts 400. Another medical tool mount 400b according to an exemplary embodiment is shown in FIG. 12. As shown, hard point 140 may be coupled to medical tool mount 400b, where medical tool mount 400b of FIG. 12 is generally distinct from medical tool mount 400 of FIGS. 2 and 7-11. Medical tool mount 400 and medical tool mount 400b may be similar in mounting hard points 140, or they may include distinct mounts. Medical tool mount 400b may be used for surgeries related to a patient's foot, for example. However, hard point 140 may be configured to be compatible with any medical tool mount 400, for example, medical tool mounts 400 shown in FIGS. 2 and 7-12. In this way, hard point 140 may be a universal interface.

Similarly, hard points 140 may be compatible with a plurality of medical tools 300. As shown in FIG. 12, a connector 302 of medical tool 300 may be coupled to hard point 140. Connector 302 may be a universal connector, as a plurality of different medical tools 300 may include the same connector 302 to couple to hard point 140. In this way, hard point 140 and medical tool mount 400, generally, may couple to multiple different medical tools 300. Medical tool 300 of FIG. 12 is distinct from medical tool 300 of FIGS. 2 and 10-11. Medical tool 300 may be used for surgeries related to a patient's foot or lower limb, for example. However, hard point 140 may be compatible with any medical tool 300 that includes the corresponding connector 302, for example, medical tools 300 shown in FIGS. 2 and 10-12. In this way as well, hard point 140 may be a universal interface.

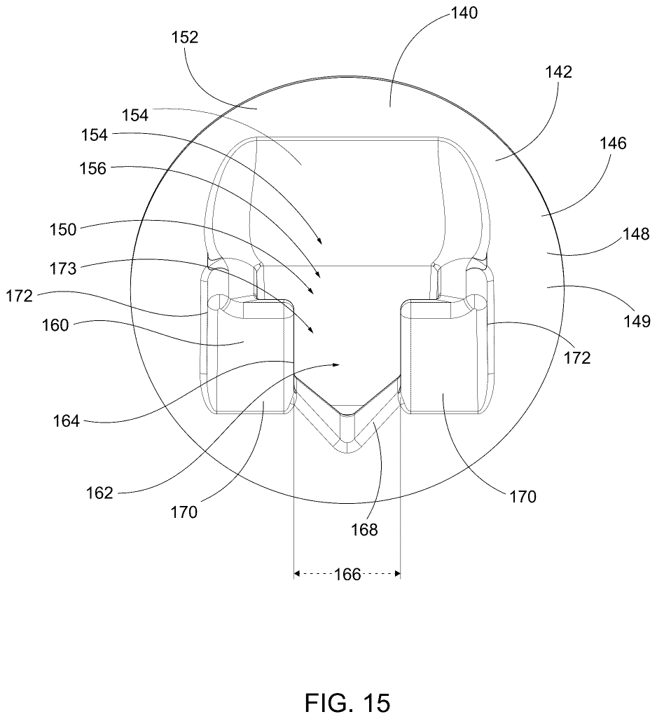

Hard point 140 may be a universal interface as it may include a shape that may receive a plurality of medical tools 300 that may be distinct. Medical tools 300 may share connector 302 that includes a shape corresponding to hard point 140 such that hard point 140 may receive each medical tool 300 that includes the corresponding connector 302. Accordingly, the interface between hard point 140 and connector 302 of medical tools 300 may be universal. In an example, medical tools 300 may fit directly over the shape of hard points 140 such that the shapes of each substantially correspond, like a “hand in glove” arrangement. The clearance between portions of hard point 140 and connector 302 of medical tools 300 may be minimal or non-existent. In other words, portions of the hard point 140 may make contact with medical tool mount 400. For example, referring to FIG. 12, a front surface 170 of hard point 140 may fit over wall 432 of medical tool mount 400b, and connector 302 may fit within a receiving cavity 150 (FIG. 15), which will be discussed further below. In addition, hard point 140 may be flexible or resilient such that hard point 140 may move with medical tools 300, or portions of medical tool mount 400. Accordingly, as medical tool mount 400 is moved, or portions of medical tool mount 400 are moved, hard point 140 may move without causing separation between hard point 140 and medical tools 300, and without bunching or folding of the hard point 140 material. As hard point 140 may create the sterile barrier between medical tool mount 400 in working space 12 (FIG. 1) and medical tool 300 in sterile space 10 (FIG. 1), and the sterile barrier between a plurality of distinct medical tool mounts 400 and medical tools 300, hard point 140 may be a universal sterile interface.

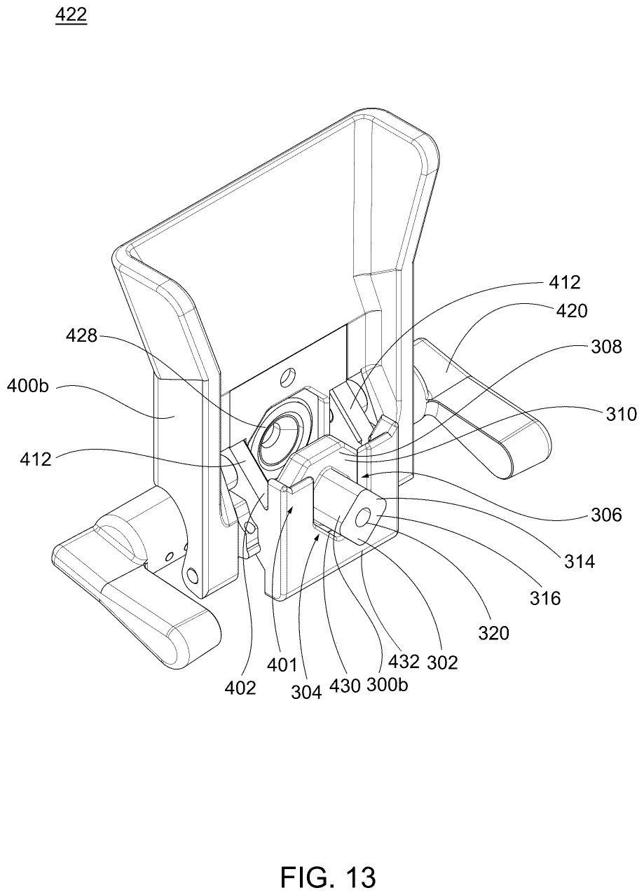

As discussed with reference to FIG. 7-11, medical tool mount 400 may be coupled to medical tool 300 through drape 100, such as through hard points 140. FIG. 12 similarly shows medical tool mount 400b coupled to medical tool 300 through hard point 140. Hard point 140 may be received by medical tool mount 400b and may releasably grip medical tool 300 to couple to medical tool 300. To facilitate this coupling, hard point 140 may include a flexible portion 146 having a flexible material 148. In this way, hard point 140 may be generally flexible, however, in some embodiments, not as flexible as remaining portions of drape 100 (FIG. 1). In addition, flexible material 148 may include a flexible material shape 149 corresponding to a shape 401 (FIG. 13) of medical tool mount 400b. Accordingly, flexible portion 146 having flexible material 148 may be flexible such that flexible portion 146 may move in conjunction with medical tool mount 400b.

In certain embodiments, flexible material 148 may releasably grip medical tool 300, such as on at least generally opposing sides, such as a first side 304 and a second side 306 opposing first side 304. Accordingly, medical tool 300 may be constrained laterally such that medical tool 300 may not move laterally when coupled to medical tool mount 400b. As will be discussed, the shape of medical tool 300 may match the shape of medical tool mount 400b where medical tool 300 is coupled to medical tool mount 400b. For example, medical tool 300 and medical tool mount 400b may include generally the same shape where they couple to one another, such as a circular shape, a round shape, or a polygonal shape. As the shapes of each of medical tool 300 and medical tool mount 400b match, the clearance between medical tool 300 and medical tool mount 400b may be minimal or non-existent, particularly in the areas where medical tool mount 400b grips or secures medical tool 300. In other words, portions of the medical tool 300 may make contact with medical tool mount 400. Therefore, medical tool 300 may not move, or may only move minimally, within medical tool mount 400b. In addition, medical tool 300 may move with medical tool mount 400, or portions of medical tool mount 400.

As shown in FIGS. 12-13, medical tool 300b may include connector 302 having first side 304 and second side 306. First side 304 and second side 306 may provide opposing surfaces for a medical tool mount to grasp the connector 302. In general, any connector for a medical tool may present an opposing first side and second side for gripping by a medical tool mount and corresponding hard point. Accordingly, flexible material 148 may releasably grip medical tool 300 having connector 302 on at least generally opposing sides of connector 302, such as first side 304 and second side 306. In this way, hard point 140 may form a sterile barrier between medical tool mount 400b disposed in working space 12 (FIG. 1) and medical tool 300 coupled to medical tool mount 400b in sterile space 10 (FIG. 1).

Referring to FIG. 13 according to an exemplary embodiment, connector 302 may include a head portion 308 extending between first side 304 and second side 306. Head portion 308 may include a head portion shape 310. Head portion shape 310 may form the shape of head portion 308 and may be circular, rectangular, or hexagonal, for example. As shown, head portion shape 310 may include one or more sides, such as about eight sides, such that head portion shape 310 is octagonal. In addition, connector 302 may include a shaft portion 314 extending from head portion 308 and between first side 304 and second side 306. Shaft portion 314 may include a shaft portion shape 316. Shaft portion shape 316 may form the cross-sectional shape of shaft portion 314 and may be circular, rectangular, hexagonal, or octagonal, for example. Accordingly, shaft portion shape 316 may include the same shape or a different shape as head portion shape 310. As shown, shaft portion shape 316 may be rectangular. Alternatively, shaft portion shape 316 may be circular or elliptical. Medical tool mount 400b may receive at least a portion of medical tool 300, such as connector 302, such as head portion 308 or shaft portion 314, such as head portion 308 and shaft portion 314.

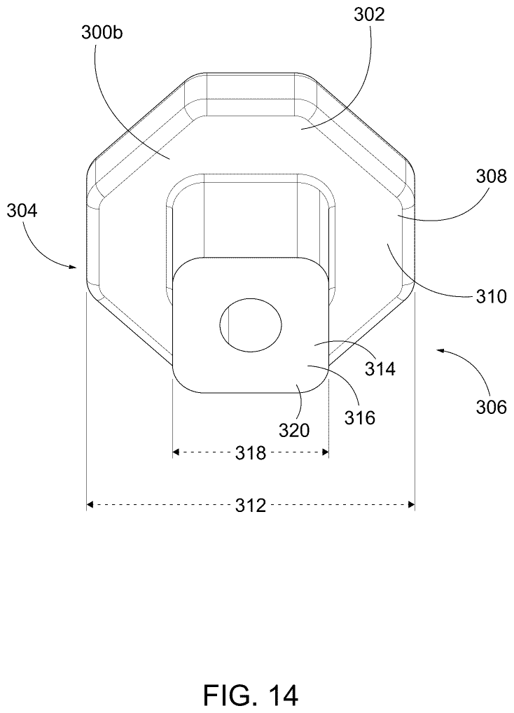

Connector 302 may be seen in FIG. 14 according to an exemplary embodiment. Shaft portion 314 may extend from head portion 308, where head portion 308 may span between first side 304 and second side 306 of connector 302 and shaft portion 314 may extend within first side 304 and second side 306. In other words, head portion 308 may be larger in diameter, or length, in comparison to shaft portion 314.

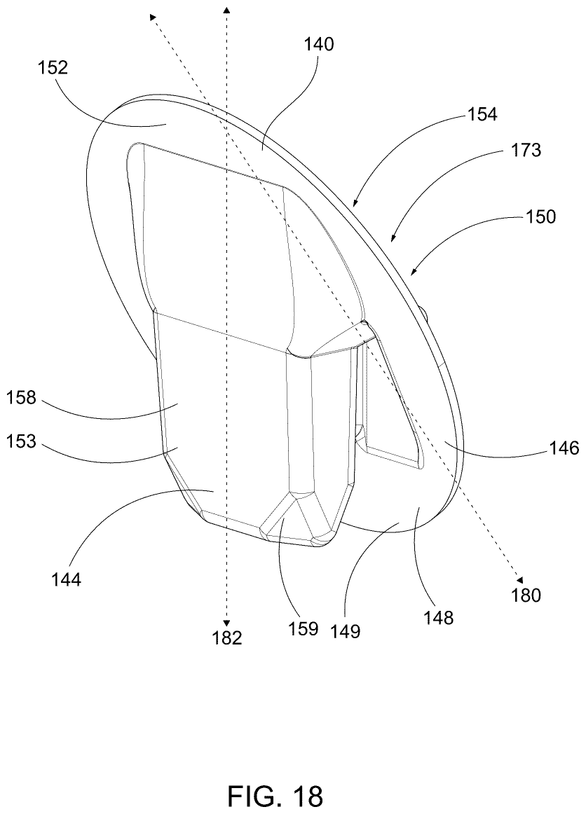

With reference to FIG. 14-15 according to exemplary embodiments, hard point 140 may releasably grip at least a portion of head portion 308 and at least a portion of shaft portion 314. As shown in FIG. 15, hard point 140 may include receiving cavity 150. Receiving cavity 150 may include one or more portions to receive medical tool 300, such as connector 302. One portion may be a recessed cavity 154. Recessed cavity 154 may be formed in a recessed pocket 153 (FIG. 18) of hard point 140. Recessed cavity 154 may receive at least a portion of connector 302 of medical tool 300, such as at least a portion of head portion 308, where head portion 308 may include a first diameter 312 and shaft portion 314 may include a second diameter 318, first diameter 312 being larger than second diameter 318.

Recessed pocket 153 (FIG. 18) may include a bottom shape 159 (FIG. 18). Recessed cavity 154 may include a recessed cavity shape 156 corresponding to bottom shape 159 and head portion shape 310 of connector 302. In other words, head portion shape 310 may correspond to the shape of the bottom of recessed pocket 153 and recessed cavity 154 such that head portion 308 fits into recessed pocket 153 within recessed cavity 154. In this way, recessed pocket 153 and recessed cavity 154 may receive at least a portion of head portion 308.

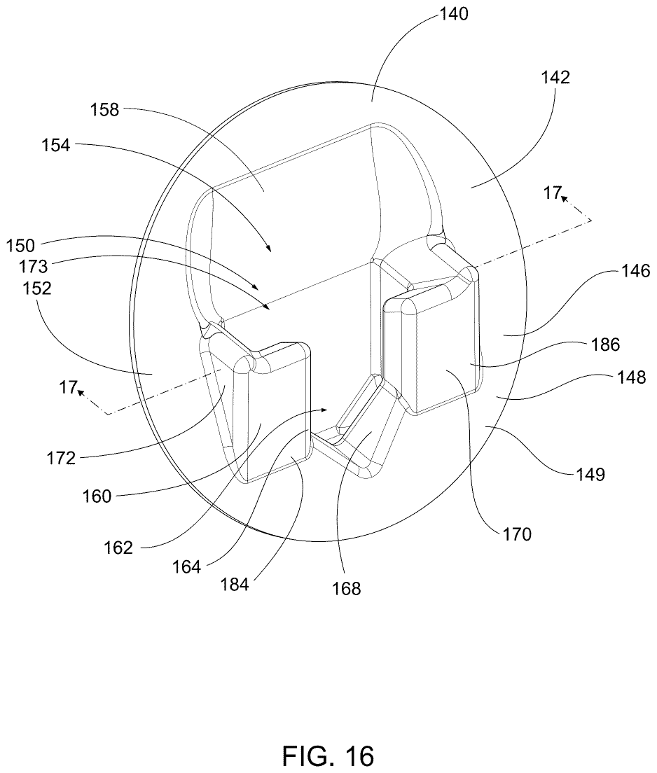

In certain embodiments, along hard point 140, may include one or more features to receive and retain at least a portion of medical tool 300b, such as at least a portion of connector 302. With reference to FIGS. 14-16 according to exemplary embodiments, hard point 140 may include a frame 160 surrounding a frame cavity 173 of receiving cavity 150 to receive and retain at least a portion of medical tool 300, such as at least a portion of connector 302, such as at least a portion of shaft portion 314. As shown in FIG. 16, frame 160 may include one or more front surfaces 170 and one or more transverse walls 172. Frame cavity 173 may extend between a rear surface 158 of hard point 140, front surfaces 170, and transverse walls 172. Frame 160 may surround, or extend from, or correspond to, recessed pocket 153. At least a portion of recessed cavity 154 may extend below frame cavity 173, as shown in FIG. 19.

Referring again to FIGS. 14-15, receiving cavity 150, in addition to recessed cavity 154 and frame cavity 173, may include a frame opening 162 through which at least a portion of medical tool 300 is received. Frame opening 162 may be formed in frame 160. In this way, front surface 170 of frame 160 may be divided into two front surfaces 170. As shown, transverse walls 172 extend outwardly from an outer frame 152 of hard point 140, which will be discussed further below. Front surfaces 170 may extend laterally inward from each transverse wall 172 ending at frame opening 162 to encircle frame cavity 173. Accordingly, with reference to FIG. 16, frame opening 162 may be the space within frame 160 between adjacent front surfaces 170.

Frame opening 162 may include a frame opening shape 164 that may be distinct from head portion shape 310. Frame opening shape 164 may instead correspond to shaft portion shape 316. Accordingly, frame opening 162 may receive shaft 314 but not head portion 308. As frame opening shape 164 may be distinct from head portion shape 310, connector 302 may be retained based on head portion 308 extending outwardly from frame opening 162 and the varying shapes of head portion shape 310 and frame opening shape 164. In other words, connector 302 may be retained transversely (into and out of the sheet), based on frame opening 162 having frame opening shape 164 being adjacent to head portion 308 of connector 302 in the transverse direction and therefore preventing movement of connector 302 out of hard point 140 in the transverse direction.

In certain embodiments, frame opening 162 may also include an edge 168. Edge 168 may be “V” shaped, for example. Alternatively, edge 168 may be straight, curved, or may include any other shape. Edge 168 may correspond to shaft portion shape 316 in certain orientations. As shown in FIGS. 13 and 15, for example, shaft portion 314 may be oriented such that a corner of shaft portion shape 316 may line up with edge 168. In this way, frame opening 162 may receive shaft 314.

In addition to the shapes of various features of hard point 140 and connector 302, retention in recessed cavity 154 may be described by the sizes of various features of hard point 140 and connector 302. Referring again to FIGS. 14-16, frame 160 may include a frame diameter 166 corresponding to second diameter 318 of shaft portion 314 such that frame opening 162 may receive shaft 314 but not head portion 308 having first diameter 312. In this way, head portion 308 may be retained in recessed cavity 154 by frame 160 when hard point 140 is coupled to medical tool mount 400b (FIG. 12) and shaft portion 314 may be retained in frame opening 162. In certain embodiments, frame diameter 166 includes a length that is less than a length of recessed cavity 154, and is substantially equal to, or greater than, second diameter 318.

In addition to the shapes and sizes of various features of hard point 140 and connector 302, the material of each may facilitate retention of medical tool 300. As discussed, hard point 140 may include flexible portion 146 having flexible material 148. Flexible portion 146 may include recessed cavity 154 and frame 160. Accordingly, recessed cavity 154 and frame 160 may include flexible material 148 such that recessed cavity 154 and frame 160 may cinch, or tighten around, or narrow around, at least a portion of connector 302, which may be a rigid component, to lock, or secure, connector 302 in hard point 140. In embodiments, medical tool mount 400b is also a rigid component. Hard point 140 may retain connector 302 by cinching around connector 302. In this way, hard point 140 may be less rigid than connector 302.

Referring to the embodiment shown in FIG. 16, hard point 140 may include outer frame 152 surrounding receiving cavity 150 and frame 160. Frame 160 may extend outwardly from outer frame 152 in a transverse direction. As shown, frame 160 may extend around frame cavity 173, and particularly front surfaces 170 and transverse walls 172 bound frame cavity 173. Frame cavity 173 is also bounded by rear surface 158 of hard point 140. In this way, at least a portion of medical tool 300 (FIG. 14), such as at least a portion of connector 302 (FIG. 14), such as at least a portion of head portion 308 (FIG. 14) may be retained in frame cavity 173 and into recessed cavity 154 extending below frame cavity 173 between outer frame 152 and frame 160.

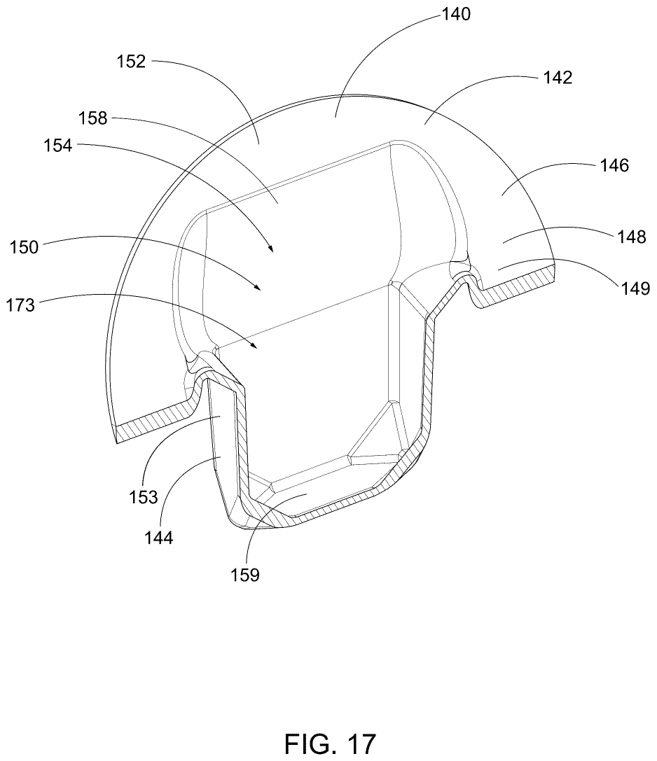

A cross section of receiving cavity 150 may be seen in FIG. 17 according to an exemplary embodiment, such that receiving cavity 150 may be visualized with frame 160 removed.

As shown, recessed cavity 154 extending below frame cavity 173 may include a shape that may be circular, rectangular, hexagonal, or octagonal, for example that is the same shape as bottom shape 159 of recessed pocket 153. Recessed pocket 153 and recessed cavity 154 may be shaped to receive at least a portion of medical tool 300, such as at least a portion of connector 302, such as at least a portion of head portion 308. Accordingly, recessed pocket 153 and recessed cavity 154 may be octagonal, for example, when head portion shape 310 (FIG. 1) is also octagonal. In other embodiments, recessed cavity 154 has a shape that corresponds to head portion 308, in at least a width dimension. The corresponding shapes may facilitate retention of at least a portion of medical tool 300, such as at least a portion of connector 302, such as at least a portion of head portion 308 in recessed cavity 154.

In certain embodiments, hard point 140 may then cinch, or tighten around, or narrow around, connector 302, which may be a rigid component, such that recessed cavity 154 becomes smaller and the clearance between recessed cavity 154 and connector 302 becomes minimal or non-existent. In this way, connector 302 may be locked, or secured, in hard point 140. In some embodiments, hard point 140 may include a first end 184 and a second end 186 As shown in FIG. 16, first end 184 may move generally toward second end 186 to releasably grip at least a portion of connector 302. Additionally or alternatively, second end 186 may move generally toward first end 184 to releasably grip at least a portion of connector 302.

In embodiments, outer frame 152 may define portions of hard point 140 that may contact sterile space 10 (FIG. 1) and working space 12 (FIG. 1). With reference to FIG. 16-18, hard point 140 may include a hard point top surface 142 that may contact sterile space 10. Hard point 140 may also include a hard point bottom surface 144 opposing hard point top surface 142 that may contact working space 12. Hard point top surface 142 and hard point bottom surface 144 may extend from outer frame 152. Bottom surface 144 may be received by medical tool mount 400b. In this way, hard point 140 may form a sterile barrier between medical tool mount 400b disposed in working space 12 and medical tool 300 coupled to medical tool mount 400b in sterile space 10.

Hard point 140 may extend beyond outer frame 152 in opposing lateral directions such that outer frame 152 is comprised by a middle portion of hard point 140. As shown in FIG. 19 according to an exemplary embodiment, frame 170, including transverse walls 172 and front surface 170, surrounding frame cavity 173 may extend outwardly from outer frame 152. Recessed pocket 153 surrounding recessed cavity 153 may extend outwardly from outer frame 152 in the opposing direction. In addition, recessed pocket 153 may extend below frame 160 along an axis 182. As shown, axis 182 and rear surface 158 of may be generally parallel to front surface 170 of frame 160.

Recessed cavity 154 extending within recessed pocket 153 may facilitate coupling of hard point 140 to medical tool mount 400b (FIG. 13). With reference to FIGS. 13 and 19, recessed cavity 154 and recessed pocket 153 may be disposed within medical tool mount 400b between walls 412 of medical tool mount 400b when hard point 140 is coupled to medical tool mount 400b. Walls 412 may be ramped, as shown in FIG. 13, to additionally facilitate this coupling. In an example, walls 412 may be ramped to guide hard point 140 and medical tool 300 into medical tool mount 400. Medical tool 300, such as outer frame 152, may include a corresponding shape. In other words, outer frame 152 may be similarly angled such that outer frame 152 may be easily positioned against walls 412 to couple hard point 140 to medical tool mount 412. As shown in FIG. 19, outer frame 152 may extend along an axis 180 such that outer frame 152 is oblique relative to front surface 170 of frame 160 and axis 182. In this way, outer frame 152 may rest along ramped walls 412 when hard point 140 is coupled to medical tool mount 400b.

Referring to FIG. 13-15, medical tool mount 400b may include a lock 420. Lock 420 may be actuated to lock, or secure, connector 302 in medical tool mount 400b. As shown in FIG. 13, lock 420 may be in a first position 422 in which connector 302 is not locked, or secured, in medical tool mount 400b. In first position 422, connector 302 may be remote from, or not proximate to, a seat 428 of medical tool mount 400b. Seat 428 may include a shape corresponding to connector 302, such as to head portion 308. Accordingly, once proximate to seat 428, connector 302 may be secured against seat 428, thereby locking connector 302 in medical tool mount 400b. Actuating lock 420 may cause lock 420 to move away from first position 422. Ramps 412 of medical tool mount 400b may retract such that transverse wall 432 retaining connector 302 is retracted, positioning connector 302 proximate to seat 428. Lock 420 may include a lever that is moved to position connector 302 proximate to seat 428. Lock 420 may additionally or alternatively include other actuation mechanisms, such as a push button, switch, handle, or other means for causing connector 302 to move proximate to seat 428.

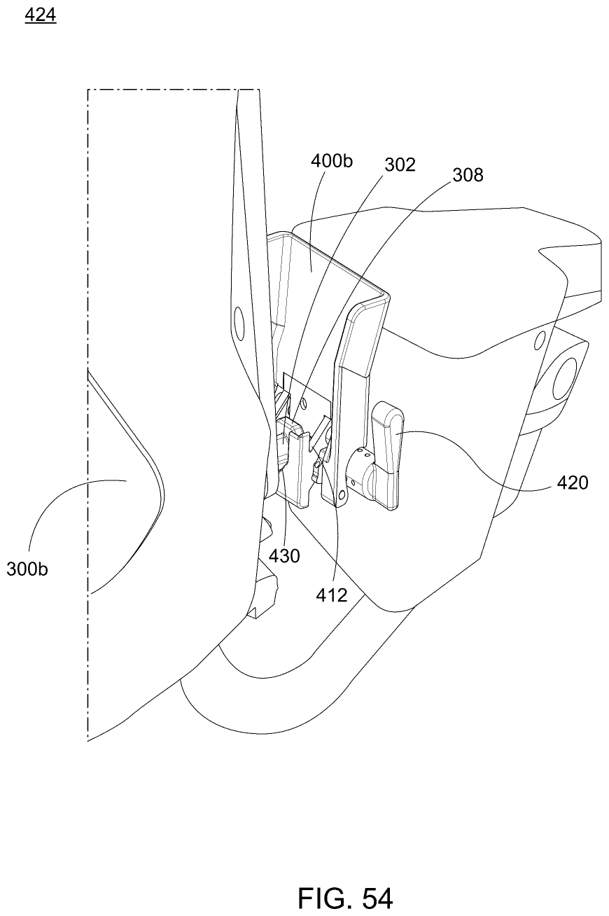

In a third position 426, shown in FIG. 54 according to exemplary embodiments, connector 302 may be proximate to seat 428. As shown, medical tool 300b may be secured to medical tool mount 400b when lock 420 is actuated and in third position 426. Here, a drape may not separate medical tool 300b and medical tool mount 400b. Instead, both medical tool 300b and medical tool mount 400b may be in sterile space 10 (FIG. 1).

In a second position 424 (not shown), lock 420 may be actuated to a position between the position shown in FIG. 13 in which lock 420 is in first position 422 and the position shown in FIG. 54 in which lock is in third position 426. In second position 424, lock 420 may be actuated to retract ramps 412 and transverse wall 432 such that hard point 140 may be proximate to seat 428. In second position 424, hard point 140 may cinch around connector 302. Here, a drape, such as drape 100, may separate medical tool 300b and medical tool mount 400b. Accordingly, medical tool 300b may be in sterile space 10 (FIG. 1) and medical tool mount 400b may be in working space 12 (FIG. 1).

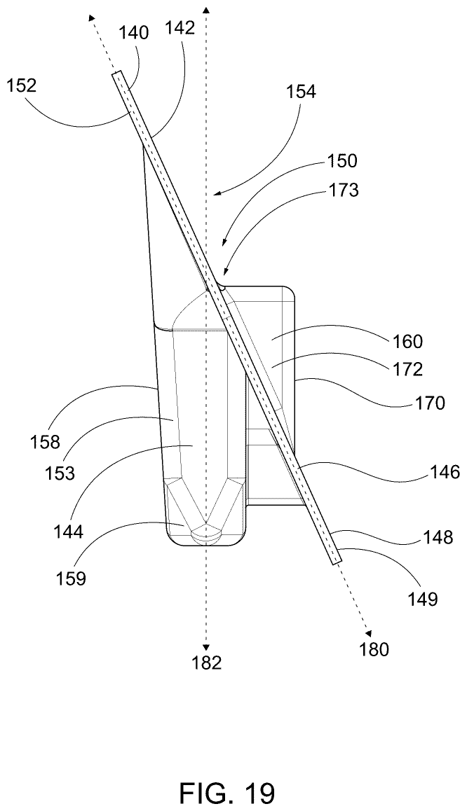

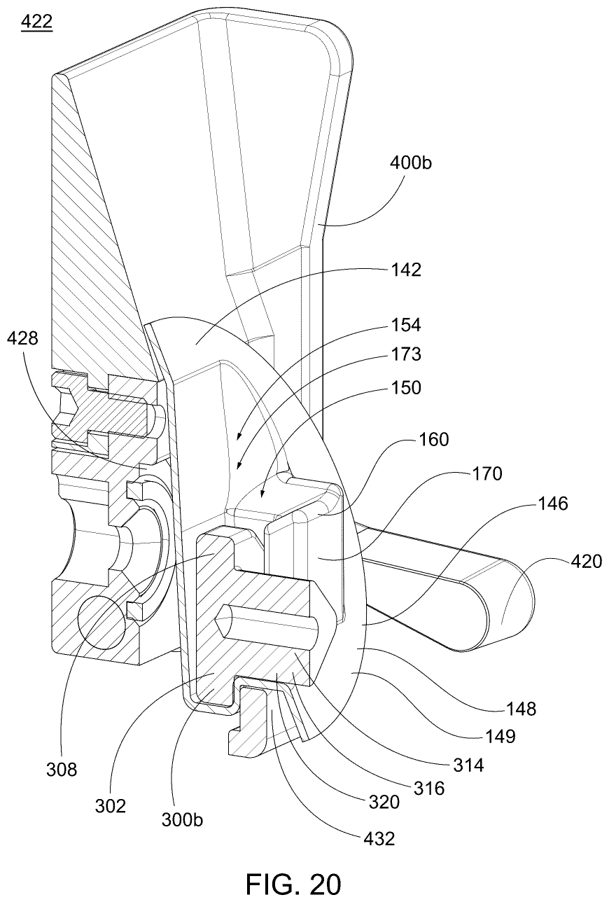

A cross-section view of hard point 140 coupled to medical tool mount 400b is shown in FIG. 20 according to an exemplary embodiment. As shown, receiving cavity 150 may receive at least a portion of medical tool 300, such as at least a portion of connector 302. At least a portion of head portion 308 may be disposed in recessed pocket 153. Accordingly, at least a portion of head portion 308 may be disposed in recessed cavity 154 of receiving cavity 150, where recessed cavity 154 may be shaped correspondingly to head portion shape 310 of head portion 308. Shaft portion 314 may be disposed in frame opening 162, which may be bounded by edge 168, frame opening shape 164 and edge 168 shaped correspondingly to shaft portion shape 316 of shaft portion 314. Frame 160 may bound head portion 308 such that connector 302 is retained in receiving opening 150. At least a portion of recessed pocket 153 and recessed cavity 154 may be disposed within medical tool mount 400b when hard point 140 is coupled to medical tool mount 400b. In addition, front surface 170 of hard point 140 may fit over wall 432 of medical tool mount 400b. Edge 168 (FIG. 16) of hard point 140 may fit over edge 430 (FIG. 13) of medical tool mount 400b. Accordingly, portions of the hard point 140 may make contact with medical tool mount 400.

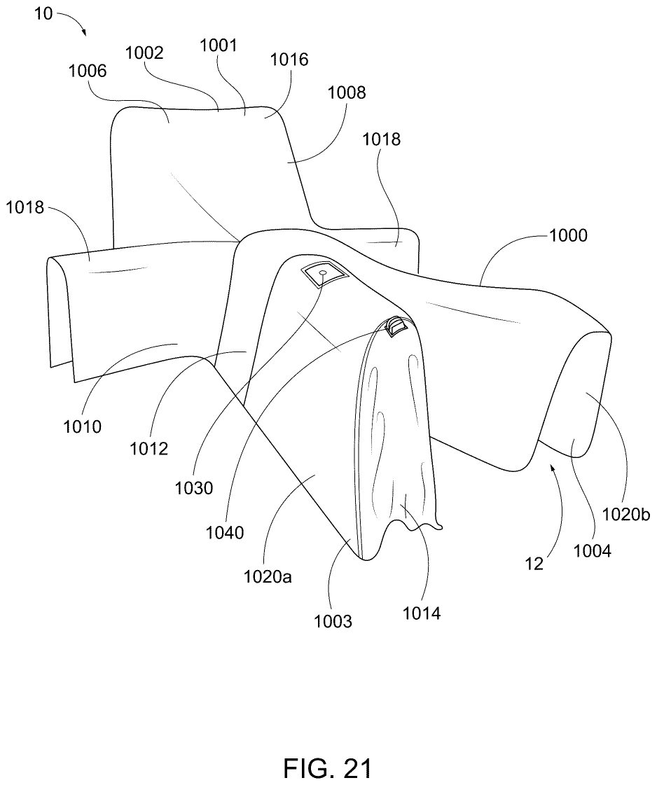

FIG. 21 shows a perspective view of a drape 1000 according to an exemplary embodiment. Drape 1000 may be substantially similar to drape 100, shown in FIG. 1. Accordingly, embodiments described with reference to drape 100 may be applicable to drape 1000 and vice versa.

Drape 1000 may be structured and configured for particular surgeries, such as knee surgery. Drape 1000 may cover a patient and various equipment, such as an operating table and related accessories, during a surgical procedure to support positioning a patient for a surgical procedure. As shown, drape 1000 may contact sterile space 10, which may include a surgical site, and working space 12. Working space 12 may include various equipment for facilitating a surgical procedure, and may also include non-operative equipment. Drape 1000 may be a boundary between sterile space 10 and working space 12 such that drape 100 separates sterile space 10 from working space 12. In this way, drape 1000 may define sterile space 10 along its top surface 1002 and working space 12 along its bottom surface 1004.

A drape material 1006 of drape 1000 may be separated into one or more sections to cover the patient and various equipment. For example, drape material 1006 may be separated into a first section 1008, a second section 1010, and a third section 1012. First section 1008 may include a head portion 1016 to cover a head of the patient. Second section 1010 may include one or more arm portions 1018 to cover arms of the patient. Third section 1012 may cover an operative leg and/or a non-operative leg, each of which may be supported under drape 1000 in working space 12 on various equipment, such as a spar or a well-leg holder. For example, an operative leg portion 1020a may cover the operative leg. A non-operative leg portion 1020b may cover the non-operative leg. Operative leg portion 1020a and non-operative leg portion 1020b may be separated as shown to allow medial access to the operative leg. First section 1008 and second section 1010 may include typical drape material, such as SMS material, as discussed above. First section 1008 may include level 3 SMS material, for example. Second section 1010 may include level 4 SMS material, such as level 3 SMS material with fluid barrier material. In embodiments, drape 100 may also include a liner 1014 coupled to third section 1012. Liner 1014 may include clear polyurethane material. Liner 1014 may be clear to facilitate access to and visualization of equipment disposed under drape 1000 in working space 12.

Drape 1000 may be structured and configured for particular surgeries, such as knee surgery. Accordingly, drape 1000 may include a knee fenestration portion 1030, such as along third section 1012 of drape 1000. Knee fenestration portion 1030 may include peel-away tape that may expose an inner adhesive for application to the patient.

As with drape top surface 102 (FIG. 1) and drape bottom surface 104 (FIG. 1) of drape 100 (FIG. 1), drape top surface 1002 and drape bottom surface 1004 may include drape material 1006. Drape material 1006 may be generally flexible material. In addition, drape 1000 may include one or more hard points 1040 to support positioning a patient for a surgical procedure. Hard point 1040 may be more rigid than drape material 1006. Hard point 1040 may include PU such that hard point 1040 is easily sterilizable. Hard point 1040 may be disposed at particular locations on drape 1000 to couple drape 1000 and/or liner 1014 to other components of the operating room environment, such as equipment in working space 12. For example, one or more hard points 1040 may be disposed along liner 1014.

Referring to FIG. 21-23 according to exemplary embodiments, hard point 1040 may couple drape 1000 to a medical tool mount 4000 and supporting one or more medical tools 3000 specific to a surgical procedure. Hard point 1040 may be disposed between medical tool mount 4000 and medical tool 300 to form a sterile barrier between medical tool mount 4000 disposed in working space 12 and medical tool 3000 coupled to medical tool mount 4000 in sterile space 10. Drape 1000 may include one or more hard points 1040 to couple to medical tool mount 4000 and medical tool 3000. Hard point 1040 may be joined with and/or integral to drape 1000, such as to at least drape top surface 1002. For example, hard point 1040 may be integral to drape top surface 1002 and drape bottom surface 1004. In other words, hard point 1040 may be formed with drape material 1006 or may be joined such that hard point 1040 is coupled to and not removable from drape 1000. Drape 1000 with drape material 1006 and one or more hard points 1040 may create the sterile barrier.

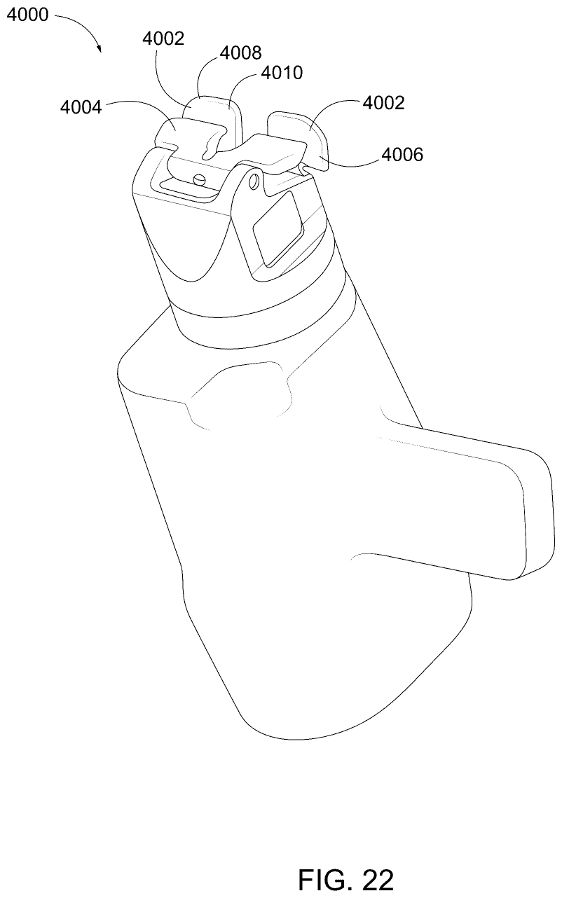

Medical tool mount 4000 may be equipment disposed in working space 12 for supporting one or more medical tools 3000 specific to a surgical procedure. FIG. 22 shows medical tool mount 4000 and medical tool 3000 according to an exemplary embodiment. Medical tool mount 4000 may include a medical tool mount shape 4002. For example, medical tool mount 4000 may include a claw 4008 having a claw shape 4010 in which a first side 4004 opposes a second side 4006. First side 4004 may move, such as rotate, toward second side 4006, such as incrementally. Additionally or alternatively, second side 4006 may move such as rotate, toward first side 4004, such as incrementally.

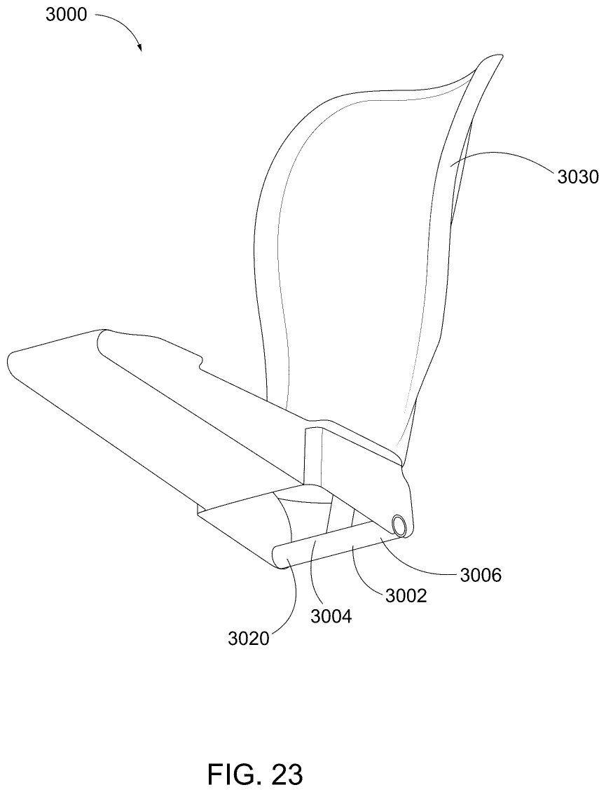

An embodiment of medical tool 3000 shown in FIG. 23, and may be a foot mount 3030, which may be used in knee surgeries. In other embodiments, medical tool 300 may be a femur hook, e.g., femur hook 340 (FIG. 2), for example. Medical tool 3000 may include a connector 3002, where connector 3002 may include an elongated member 3020, as shown. At least a portion of connector 3002 including elongated member 3020 may be received by medical tool mount 4000. Medical tool mount 4000 may be structured to support medical tool 3000, such as foot mount 3030. An example of a medical tool mount 4000 is a manipulator arm. Medical tool 3000 may couple to medical tool mount 4000 by, for example, medical tool mount 4000 gripping or receiving medical tool 3000 via one or more sides of medical tool 3000, such as one or more sides of a connector 3002 of medical tool 3000, such as a first side 3004 or a second side 3006 opposing first side 3004 of connector 3002, such as first side 3004 and second side 3006.

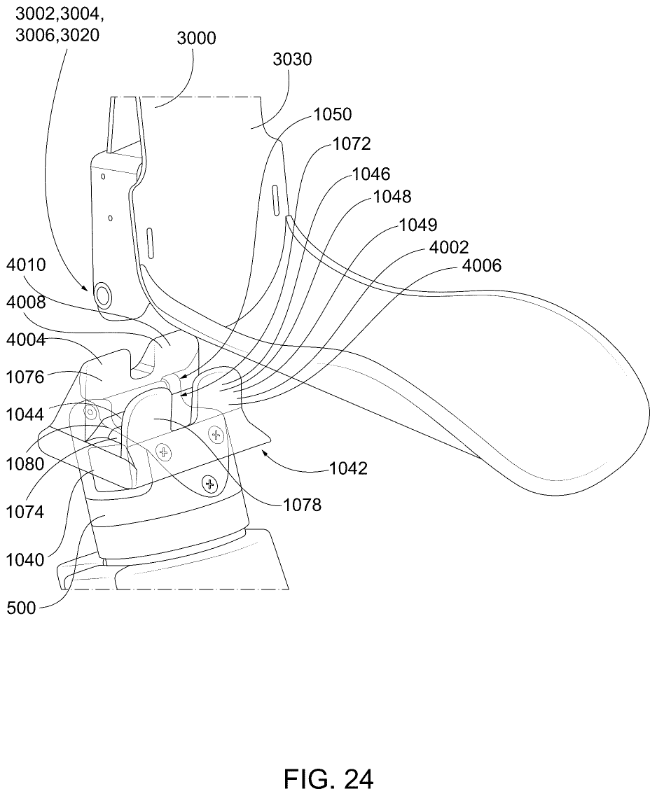

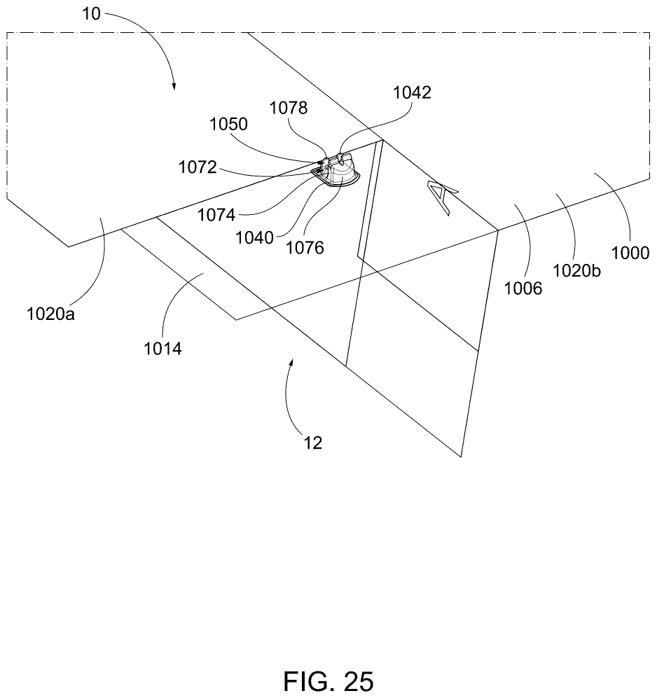

Medical tool mount 4000 may receive one or more hard points 1040 along drape 1000, as shown with reference to FIGS. 24-25 according to exemplary embodiments. Hard point 1040, while coupled to medical tool mount 4000, may releasably grip at least a portion of medical tool 3000 to couple to medical tool 3000. Hard point 1040 may be disposed above medical tool mount 4000 such that hard point 1040 may form a sterile barrier between medical tool mount 4000 shown disposed in working space 12 and sterile space 10. While medical tool mount 4000 shown in FIG. 22 is disposed in working space 12, medical tool 3000 shown in FIG. 23 may be disposed in sterile space 10. Hard point 1040 may be disposed between medical tool mount 4000 and medical tool 3000 to form a sterile barrier between medical tool mount 4000 disposed in working space 12 and medical tool 3000 coupled to medical tool mount 4000 in sterile space 10.

Hard point 1040 may be coupled to medical tool mount 4000 and medical tool 3000 such that medical tool mount 4000 and medical tool 3000 are coupled to one another through hard points 1040. In other words, medical tool mount 4000 may be coupled to medical tool 3000 through drape 1000, such as through hard points 1040. The coupling may be secured as shown such that sterile space 10 is maintained for the duration of a surgical procedure. Hard point 1040 may secure both medical tool mount 4000 and medical tool 3000 such that decoupling is prevented and risk of sterile space 10 being contaminated or non-sterile is mitigated. Drape 1000 with hard point 1040, therefore, may create the sterile barrier between medical tool mount 4000 in working space 12 and medical tool 3000 in sterile space 10. The sterile barrier remains even when medical tool 3000 is decoupled from medical tool mount 4000, allowing medical tool 3000 to be coupled and decoupled from medical tool mount 4000 a plurality of times, all while maintaining a sterile barrier.

Hard point 1040 may be received by medical tool mount 4000 and may releasably grip at least a portion of medical tool 3000 to couple to medical tool 3000. To facilitate this coupling, hard point 1040 may include a flexible portion 1046 having a flexible material 1048. In this way, hard point 1040 may be generally flexible, however, in certain embodiments, it may not be as flexible as remaining portions of drape 1000. Flexible material 1048 may include flexible material shape 1049 corresponding to medical tool mount shape 4002 of medical tool mount 4000. Accordingly, flexible portion 1046 having flexible material 1048 may be flexible such that flexible portion 1046 may move in conjunction with medical tool mount 4000. Medical tool mount 4000 may include claw 4008 having claw shape 4010 in which first side 4004 may move toward second side 4006, such as incrementally to releasably grip at least a portion of medical tool 3000, such as a connector 3002 of medical tool 3000, such as first side 3004 or second side 3006 of connector 3002, such as first side 3004 and second side 3006. In other words, claw 4008 may be a clamp that may hold connector 3002. Claw 4008 may rotate inward. For example, first side 3004 and second side 3006 may rotate toward one another, or first side 3004 may rotate toward second side 3006, or second side 3006 may rotate toward first side 3004 to rotate claw 4008 inward. Claw 4008 may include an opening to allow connector 3002 to be inserted. Accordingly, claw 4008 may include an opening size greater than a width or diameter of connector 3002.

Any opposing side may be gripped of connector 3002. For example, connector 3002 may be cylindrical. Accordingly, connector 3002 may be gripped along two or more portions that may be curved and form sides of connector 3002. In another example, connector 3002 may be hexagonal, rectangular, polygonal, or any other shape. Accordingly, connector 3002 may be gripped along two or more planar portions that may form sides of connector 3002. First side 4004 may move incrementally toward second side 3006 such that the grip force exerted on medical tool 3000 by medical tool mount 4000 may be varied to accommodate insertion of medical tool 3000 and relative motion between medical tool 3000 and medical tool mount 4000. Additionally or alternatively, second side 4006 may move toward first side 4004, such as incrementally, to releasably grip at least a portion of medical tool 3000. In this way, hard point 1040 may form a sterile barrier between medical tool mount 4000 disposed in working space 12 and medical tool 3000 coupled to medical tool mount 4000 in sterile space 10.

As hard point 1040 may move in conjunction with medical tool mount 4000, hard point 1040, with reference to FIG. 24, flexible material shape 1049 of hard point 1040 may correspond to medical tool mount shape 4002, such as claw shape 4010. In addition, as medical tool mount 4000 may include one or more sides, such as first side 4004 and second side 4006, and hard point 1040 may move in conjunction with medical tool mount 4000, hard point 1040 may also include one or more sides that move in conjunction with the one or more sides of medical tool mount 4000. For example, hard point 1040 may include a first end 1076 and a second end 1078 opposing first end 1076. First end 1076 may move such as rotate, toward second end 1078, such as incrementally. Accordingly, first end 1076 and second end 1078 may adjust a grip on medical tool 3000 such that relative movement of medical tool 3000 may occur with friction from the grip. Additionally or alternatively, second end 1078 may move such as rotate, toward first end 1076, such as incrementally. Hard point 1040 may be less rigid than connector 3002. In this way, hard point 1076 may releasably grip at least a portion of medical tool 3000, such as at least a portion of connector 3002 of medical tool 3000, such as first side 3004 or second side 3006 of connector 3002, such as first side 3004 and second side 3006, to couple medical tool 3000 to medical tool mount 4000.

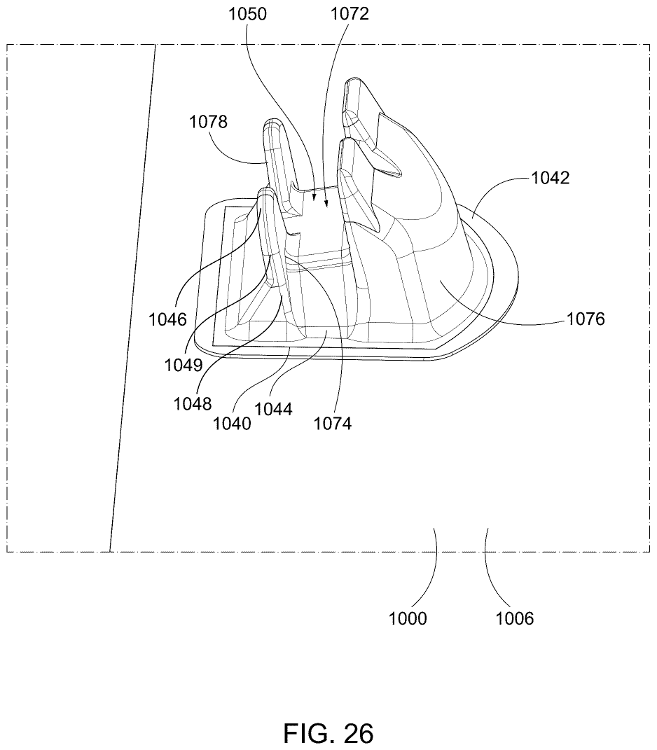

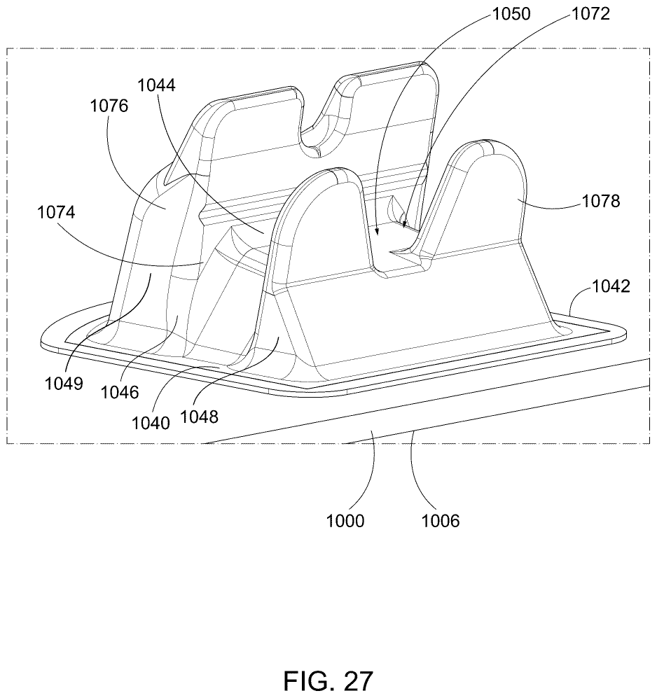

Referring to FIGS. 26-27 according to exemplary embodiments, hard point 1040 may include a gripping portion 1074 to releasably grip at least a portion of medical tool 3000 (FIG. 23), such as connector 3002 (FIG. 23) of medical tool 3000. Gripping portion 1074 may grip at least a portion of medical tool 3000, such as at least a portion of connector 3002 on at least generally opposing sides. For example, gripping portion 1074 may grip at least a portion of medical tool 3000, such as at least a portion of connector 3002, on two or more sides. Hard point 1040, such as gripping portion 1074, may move relative to drape material 1006 or medical tool 3000 to incrementally grip at least a portion of medical tool 3000, such as at least a portion of connector 3002. In other words, hard point 1040, such as gripping portion 1074, may move relative to drape material 1006 or medical tool 3000 to adjust a grip of the coupling of medical tool 3000, such as connector 3002, to medical tool mount 4000. Gripping portion 1074 may include first end 1076 and second end 1078 to releasably grip at least a portion of medical tool 3000. For example, second end 1078 may move generally toward first end 1076, such as incrementally, to releasably grip at least a portion of medical tool 3000. Additionally or alternatively, first end 1076 may move generally toward second end 1078, such as incrementally, to releasably grip at least a portion of medical tool 3000.

As shown, hard point 1040 may include a channel 1072 extending between first end 1076 and second end 1078. Channel 1072 may correspond to the opening between first side 4004 (FIG. 24) and second side 4006 (FIG. 24) of medical tool mount 4000 (FIG. 24). Channel 1072 may receive at least a portion of medical tool 3000. For example, channel 1072 may receive at least a portion of connector 3002 (FIG. 23). Connector 3002 may include an elongated member 3020 such that channel 1072 may receive at least a portion of connector 3002 including elongated member 3020. Channel 1072 may be formed between first end 1076 and second end 1078, which may extend from a bottom surface 1044.

Referring to FIGS. 24 and 26, the width and length of channel 1072 may allow rotation of medical tool 3000 within hard point 1040 without medical tool 3000 decoupling from channel 1072. In addition, channel 1072 may include rounded sides between first end 1076 and second end 1078. Accordingly, as medical tool 3000 is rotated laterally, or into and out of the page in FIG. 24, channel 1072 may allow medical tool 3000 to roll along channel 1072 without decoupling from hard point 1040.

As shown, first end 1076 may extend above second end 1078 relative to bottom surface, as shown. First end 1076 may also include a curvature. In comparison to second end 1078, first end 1076 may extend further upward and transversely, and may provide a rounded surface. First end 1076 may protrude to provide interference to medical tool 3000 as medical tool 3000 rotates relative to hard point 1040. For example, as medical tool 3000 is rotated backwards, or to the left of the page in FIG. 24, medical tool 3000 may eventually rotate against first end 1076. First end 1076, providing interference during rotation of medical tool 3000 and a curvature on which to rotate, may provide support to medical tool 3000, preventing medical tool 3000 from decoupling from hard point 1040.

Referring to FIG. 24, hard point 1040 may include a hard point top surface 1042 that may contact sterile space 10. Hard point 1040 may also include a hard point bottom surface 1044 opposing hard point top surface 1042 that may contact working space 12. Bottom surface 1044 (FIG. 24) may be received by medical tool mount 4000. In this way, hard point 1040 may form a sterile barrier between medical tool mount 4000 disposed in working space 12 and medical tool 3000 coupled to medical tool mount 4000 in sterile space 10.

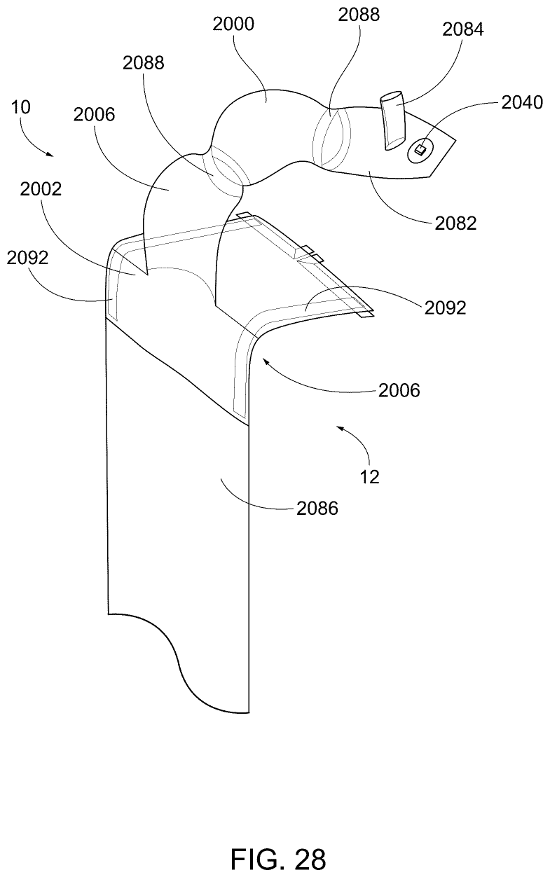

FIG. 28 shows a perspective view of a drape 2000 according to an exemplary embodiment. Drape 2000 may be substantially similar to drape 100, shown in FIG. 1, and drape 1000, shown in FIG. 21. Accordingly, embodiments described with reference to drape 100 and drape 1000 may be applicable to drape 2000 and vice versa.

Drape 2000 may be structured and configured for particular surgeries, such as hip or knee surgery In these and other surgeries, a manipulator arm may facilitate positioning one or more of a patient or medical equipment. Drape 2000 may be a manipulator arm surgical drape that may receive at least a portion of a manipulator arm, or articulating arm, or other robotic arm. As shown, drape 2000 may contact sterile space 10, which may include a surgical site, and working space 12, which may include various equipment for facilitating a surgical procedure.

In certain embodiments, drape material 2006 of drape 2000 may be separated into one or more sections to cover the patient and various equipment, such as a manipulator arm. For example, drape material 2006 may be separated into an arm drape 2082 and a table drape 2086 coupled to arm drape 2082. Arm drape 2082 may cover a medical tool mount, such as a manipulator arm. Table drape 2086 may cover at least a portion of a surgical table. As shown, arm drape 2082 may include a pouch 2084. Pouch 2084 may extend from arm drape 2082, such as extend perpendicularly from arm drape 2082. Arm drape 2082 may include a fabric material. In addition, table drape 2086 may include a fabric material. Alternatively, arm drape 2082 and table drape 2086 may include different materials.

As with drape top surface 102 (FIG. 1) and drape bottom surface 104 (FIG. 1) of drape 100 (FIG. 1), and drape top surface 1002 (FIG. 21) and drape bottom surface 1004 (FIG. 21) of drape 1000 (FIG. 21), drape 2000 may include drape material 2006. Drape material 1006 may be generally flexible material. In addition, drape 2000 may include one or more hard points 2040 to support positioning a patient or a medical tool for a surgical procedure. Hard point 2040 may be more rigid than drape material 2006. Hard point 2040 may be disposed at particular locations on drape 2000 to couple drape 2000 to other components of the operating room environment, such as equipment in working space 12. For example, one or more hard points 2040 may be disposed along arm drape 2082. In an example, pouch 2084 may include one or more hard points 2040. Any portion of arm drape 2082 may include one or more hard points 2040 in addition to the locations with hard points 2040 shown in the figures. In certain embodiments, hard point 2040 may include any embodiment of the hard points discussed herein. In other embodiments, hard point 2040 may be similar to hard point 140 and may include one or more features of hard point 140 (FIG. 1) described herein. Additionally or alternatively, Hard point 2040 may be similar to hard point 1040 (FIG. 21) and may include one or more features of hard point 140 (FIG. 1) and may include one or more features of hard point 1040 described herein.

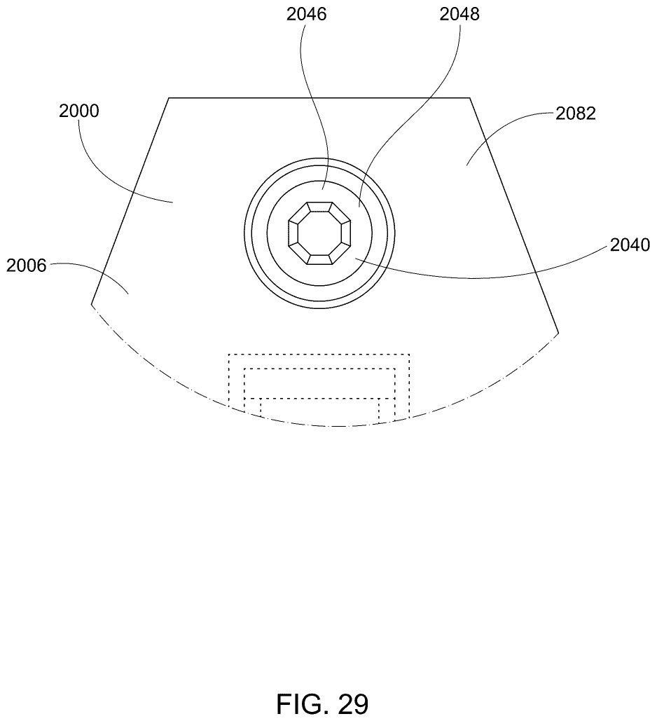

Referring to FIG. 29, according to an exemplary embodiment, hard point 2040 may couple drape 2000 to a medical tool mount, such as a manipulator arm, for positioning a patient for a surgical procedure and/or supporting one or more medical tools specific to a surgical procedure to position a patient for the surgical procedure. Hard point 2040 may be disposed between the medical tool mount and the medical tool to form a sterile barrier between the medical tool mount disposed in working space 12 and the medical tool coupled to the medical tool mount in sterile space 10. Drape 2000 may include one or more hard points 2040 to couple to the medical tool mount and the medical tool. Hard point 2040 may be integral to and/or coupled with drape 2000, such as to at least drape top surface 2002. For example, hard point 2040 may be integral to drape top surface 2002 and drape bottom surface 2004. In other words, hard point 2040 may be formed with drape material 2006 or may be coupled such that hard point 1040 is coupled to and not removable from drape 2000. Drape 2000 with drape material 2006 and one or more hard points 2040 may create the sterile barrier.

The medical tool mount for drape 2000 may be similar to medical tool mount 400 (FIG. 2) and medical tool mount 4000 (FIG. 21), among other medical tool mounts, and may include one or more features of medical tool mount 400 and medical tool mount 4000 described herein. In addition, the medical tool for drape 2000 may be similar to medical tool 300 or medical tool 3000, among other medical tools, and may include one or more features of medical tool 300 (FIG. 2) or medical tool mount 4000 (FIG. 22) described herein. For example, the medical tool mount for drape 2000 may include a medical tool mount shape. Similar to medical tool mount 400 or medical tool mount 4000, the medical tool mount for drape 2000 may receive one or more hard points 2040 along drape 2000. In an example, the medical tool mount for drape 2000 may be similar to medical tool mount 400. The medical tool received by hard point 2040 along drape 2000 may be similar to medical tool 300. Accordingly, for example, hard point 2040 may receive connector 302 (FIG. 13), which may be a universal connector.

The medical tool mount for drape 2000 may receive one or more hard points 2040 along drape 2000. Hard point 2040, while coupled to a medical tool mount, such as a manipulator arm, may releasably grip a medical tool to couple to the medical tool. Referring to FIG. 28-29, hard point 2040 may be disposed above the medical tool mount (when manipulator arm is extended vertically) such that hard point 2040 may form a sterile barrier between the medical tool mount disposed in working space 12 and sterile space 10. While the medical tool mount is disposed in working space 12, the medical tool may be disposed in sterile space 10. Hard point 2040 may be disposed between the medical tool mount and the medical tool to form a sterile barrier between the medical tool mount disposed in working space 12 and the medical tool coupled to the medical tool mount in sterile space 10.

Hard point 2040 may be coupled to a medical tool mount, such as a manipulator arm, such that the medical tool mount and a medical tool are coupled to one another through hard points 2040. In other words, the medical tool mount may be coupled to the medical tool through drape 2000, such as through hard points 2040. The coupling may be secured as shown such that sterile space 10 is maintained for the duration of a surgical procedure. Hard point 2040 may secure both the medical tool mount and the medical tool such that decoupling is prevented and risk of sterile space 10 being contaminated or non-sterile is mitigated. Drape 2000 with hard point 2040, therefore, may create the sterile barrier between the medical tool mount in working space 12 and the medical tool in sterile space 10. In certain embodiments, sterile space 10 is maintained for the duration of a surgical procedure even if medical tools, such as medical tool 300 (FIG. 2) or medical tool mount 4000 (FIG. 22), are decoupled from hard points 2040 and medical tool mounts, such as medical tool mount 400 (FIG. 2) and medical tool mount 4000 (FIG. 21). In other words, medical tools may be coupled and decoupled from hard points 2040 a plurality of times without breaching the sterile barrier formed by drape 2000. Hard points 2040 may be flexible or resilient such that hard point 2040 may be removed without causing drape 2000 to tear.

Hard point 2040 may be received by a medical tool mount, such as a manipulator arm, and may releasably grip a medical tool to couple to the medical tool. To facilitate this coupling, hard point 2040 may include a flexible portion 2046 having a flexible material 2048. In this way, hard point 2040 may be generally flexible, however, in certain embodiments, not as flexible as remaining portions of drape 2000. Flexible material 2048 may include flexible material shape 2049 corresponding to a medical tool mount shape of the medical tool mount. Accordingly, flexible portion 2046 having flexible material 2048 may be flexible such that flexible portion 2046 may move in conjunction with the medical tool mount. In this way, hard point 2040 may form a sterile barrier between the medical tool mount disposed in working space 12 and the medical tool coupled to the medical tool mount in sterile space 10. As hard point 2040 may move in conjunction with a medical tool mount, such as a manipulator arm, hard point 2040, flexible material shape 2049 of hard point 2040 may correspond to a medical tool mount shape of the medical tool mount.

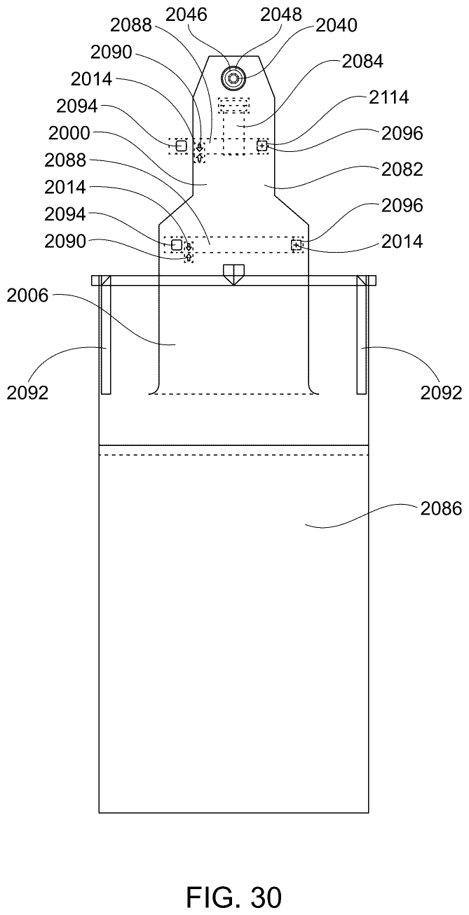

Drape 2000 may secure to a medical tool mount, such as a manipulator arm, using hard point 2040 and additionally or alternatively, one or more films 2088, as shown in FIGS. 28 and 30, according to exemplary embodiments. Films 2088 may extend around arm drape 2082 at one or more portions of arm drape 2082. For example, about two films 2088 may extend around arm drape 2082 at two separate portions of arm drape 2082. Films 2088 may fasten or secure arm drape 2082 to at least a portion of equipment that drape 2000 is covering, such as a medical tool mount, such as a manipulator arm. With reference to FIG. 28, arm drape 2082 may extend from table drape 2086 to cover the medical tool mount extending from the surgical table covered by table drape 2086. In this way, films 2088 may fasten or secure arm drape 2082 to the medical tool mount extending from the surgical table. Films 2088 may cinch arm drape 2082 to fasten or secure arm drape 2082 to the medical tool mount.

Referring to FIGS. 28 and 30, according to exemplary embodiments, drape 2000 may include one or more tapes 2092. Tapes 2092 may extend alone one or more sides of drape 2000, such as one or more sides of table drape 2086. Tapes 2092 may fasten or secure drape 2000 to at least a portion of equipment or the surgical table that drape 2000 covers. For example, tapes 2092 may extend along table drape 2086 to secure drape 2000, such as table drape 2086, to the surgical table, or another drape.

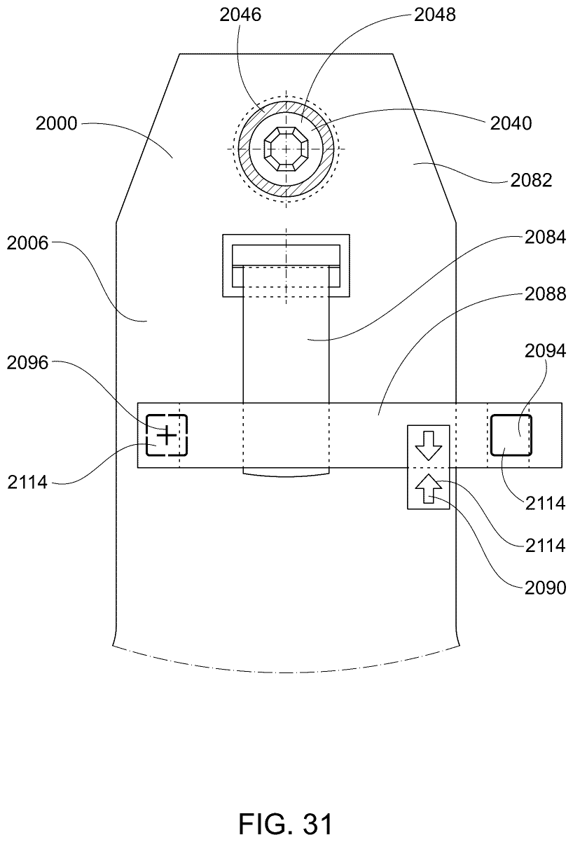

Drape 2000 may also include one or more labels 2114. Labels 2114 may indicate a step for unfolding drape 2000. In other words, labels 2114 may provide instructions on how to unfold drape 2000 once it is taken out of a packaging in order to protect or maintain the sterility of drape 2000 as it is deployed from a folded configuration. As shown in FIGS. 30-31, according to exemplary embodiments, each of one or more films 2088 may include one or more labels 2114. An upper film 2088 on arm drape 2082 may include an “apply here” label 2096. Additionally, an upper film 2088 may include an “apply” label 2096. An upper film 2088 may also include a “tear here” label 2090. A lower film 2088 may include “apply” label 2096. Additionally, a lower film 2088 may include “apply” label 2096. “Apply” labels may indicate where to fasten or secure arm drape 2082 via film 2088 to secure arm drape 2082 to at least a portion of equipment that drape 2000 is covering, such as a medical tool mount, such as a manipulator arm.

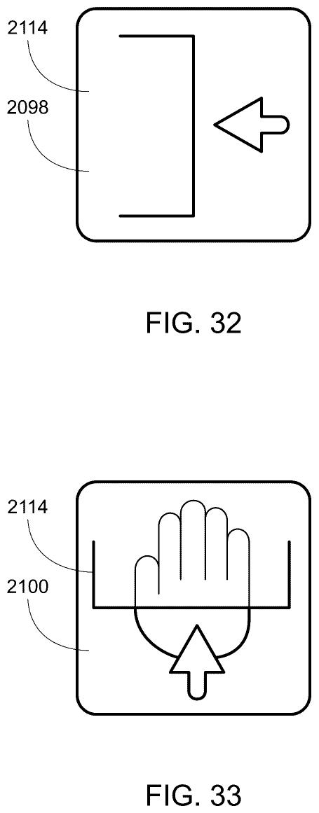

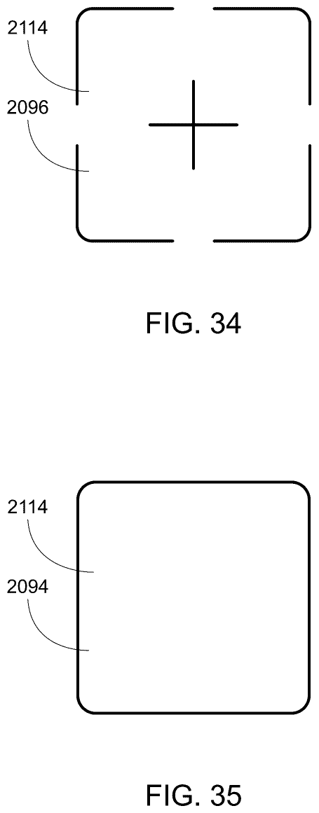

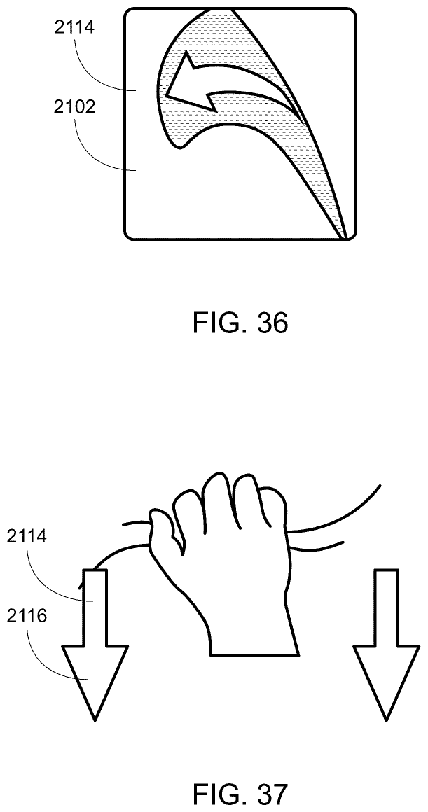

Various labels 2114 for folding drape 2000 are shown in FIGS. 32-37, according to exemplary embodiments. FIG. 32 shows an insert hand label 2100. FIG. 33 shows apply here label 2096. FIG. 35 shows apply label 2094. FIG. 36 shows a peel here label 2102. FIG. 37 shows a breakaway label 2116.

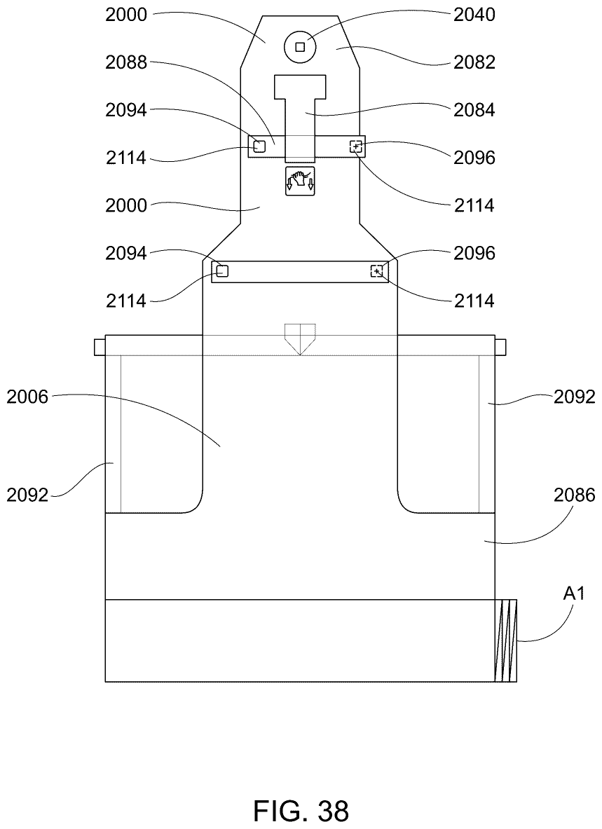

A method of folding and packaging drape 2000 may utilize one or more steps shown in FIGS. 38-51, according to exemplary embodiments. The side of drape 2000 that contacts sterile space 10 (FIG. 28) is folded inward to avoid or limit exposure to working space 12 (FIG. 28) when unpackaged.

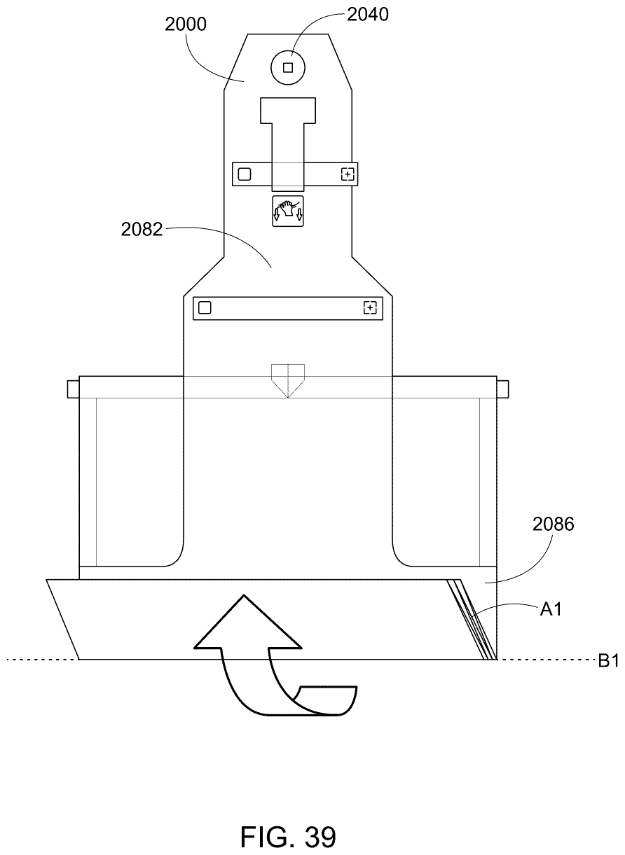

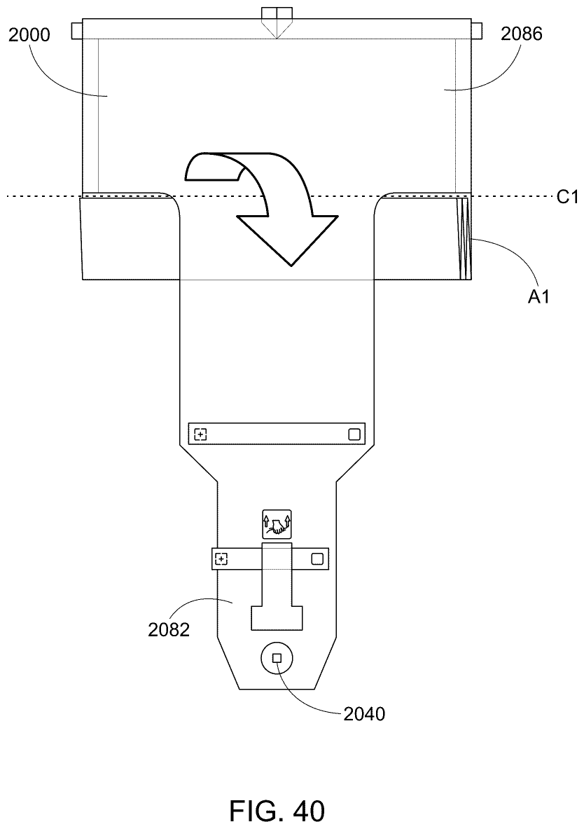

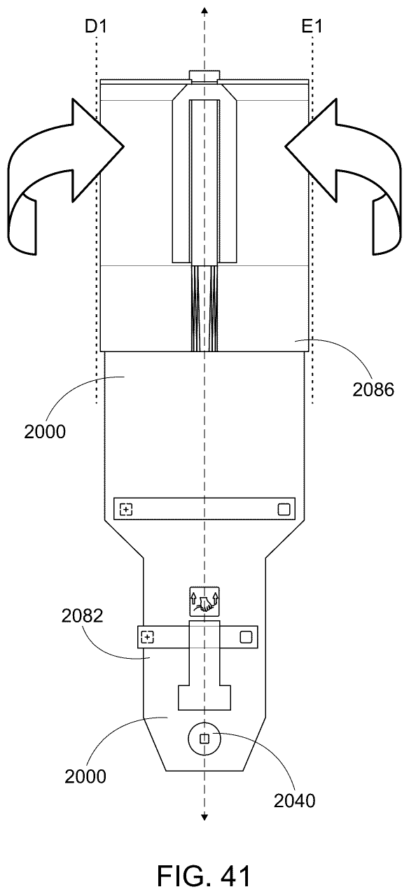

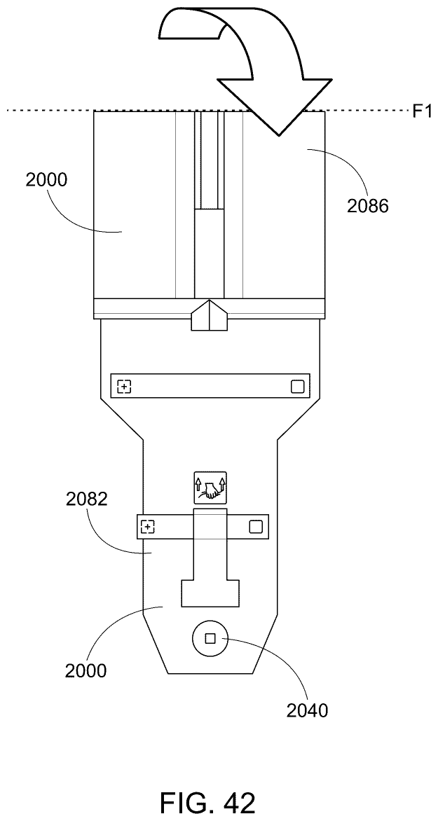

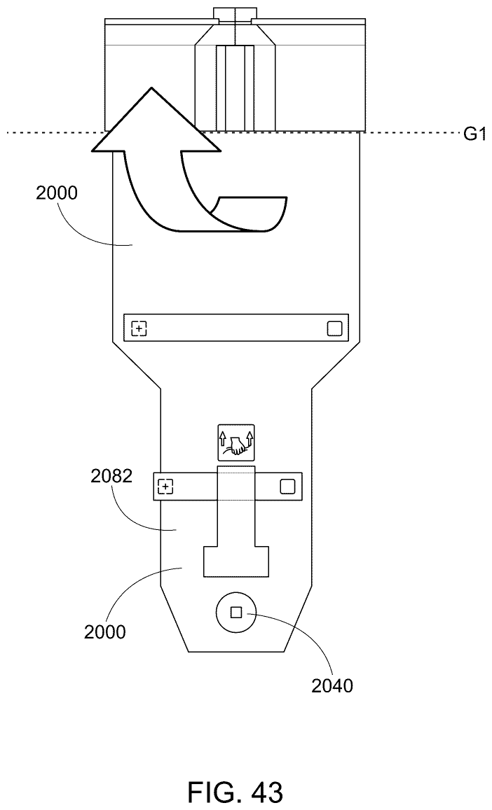

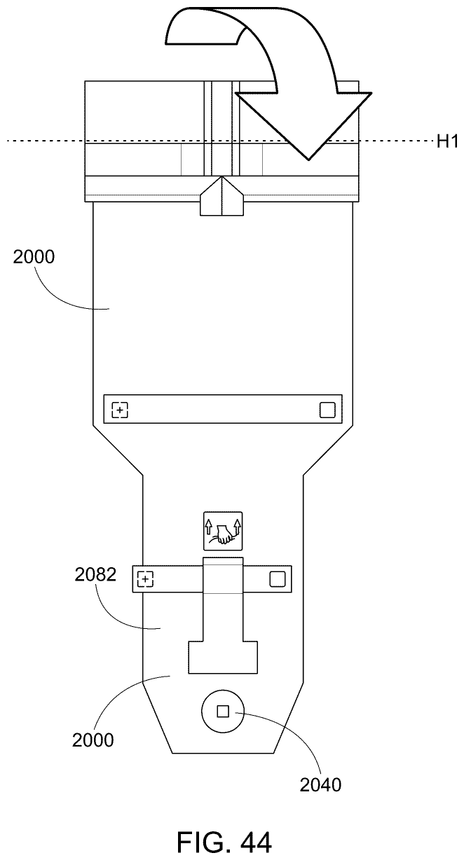

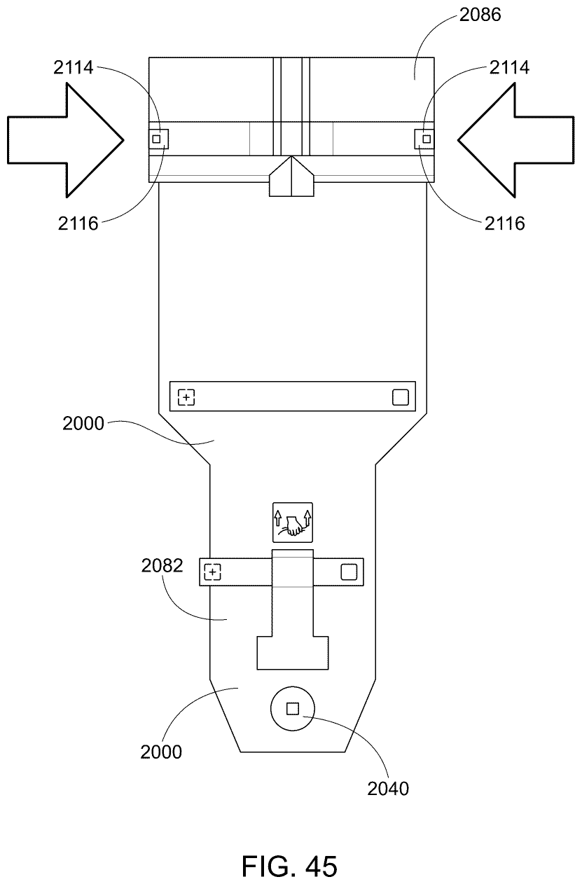

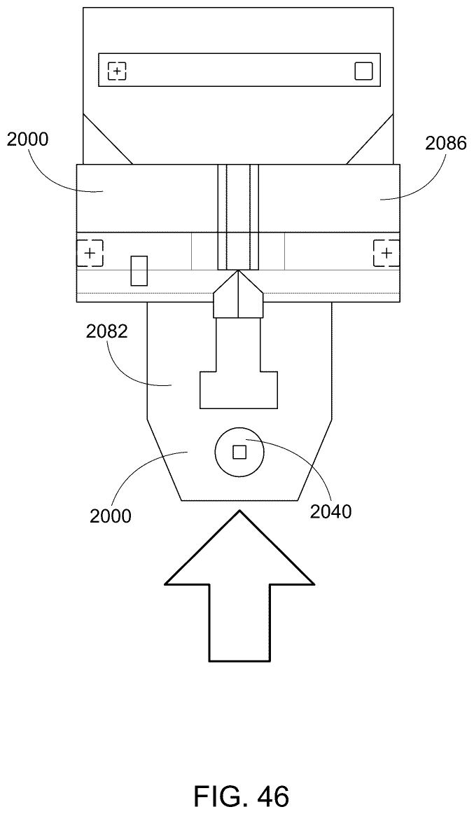

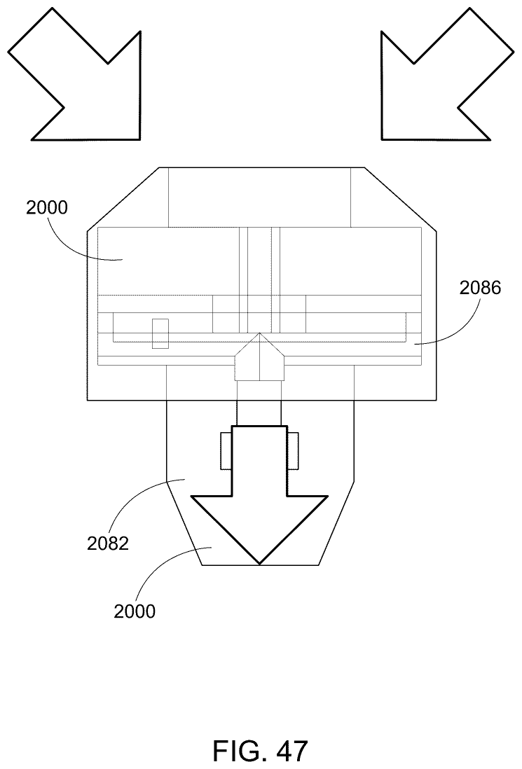

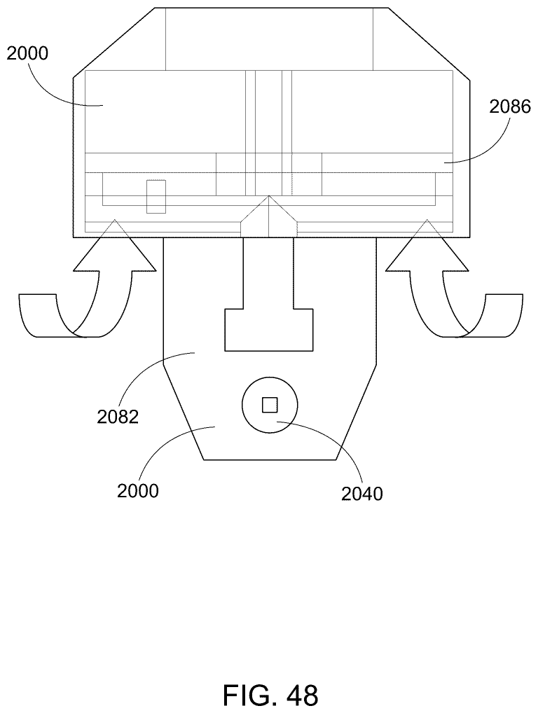

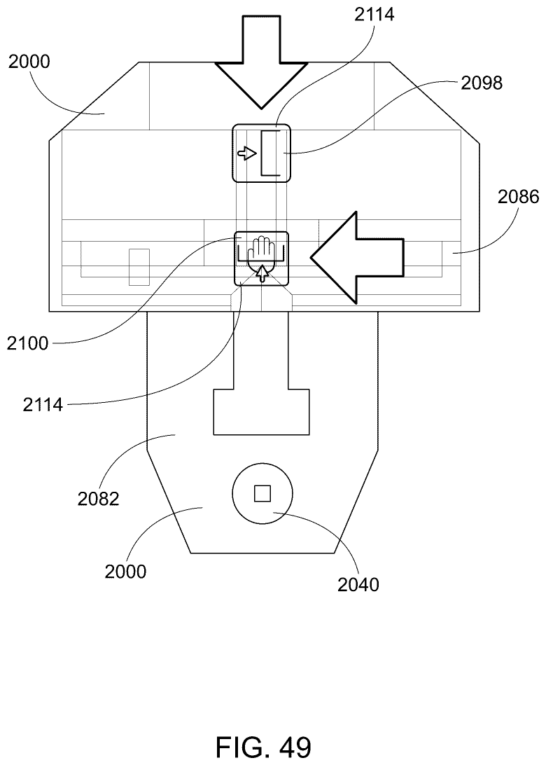

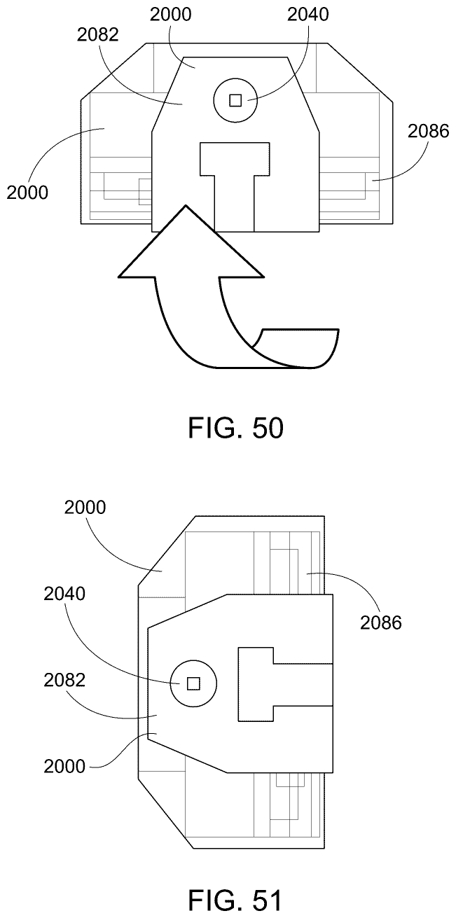

As shown in FIG. 38, the method may include accordion folding arm drape 2000, such as at least a portion of table drape 2086, in a fold A1. In other words, drape 2000, such as at least a portion of table drape 2086, may be folded back and forth with one or more folds to form one or more bellows, or pleats. As shown in FIG. 39, the method may include folding drape 2000, such as at least a portion of table drape 2086, such as accordion fold A1, along a line B1. As shown in FIG. 40, the method may include folding drape 2000, such as at least a portion of arm drape 2082 over fold A1, such as along a line C1. In other words, drape 2000, such as at least a portion of arm drape 2082 may be folded along line C1 such that arm drape 2082 is folded over accordion fold A1. As shown in FIG. 41, the method may include folding sides of drape 2000, such as at least a portion of table drape 2086 in a fold along a line D1 and a line E1. The sides may be folded toward a centerline 2116. As shown in FIG. 42, the method may include folding drape 2000, such as at least a portion of table drape 2086, along a line F1 toward arm drape 2082. As shown in FIG. 43, the method may include folding drape 2000, such as at least a portion of table drape 2086, along a line G1 toward another portion of table drape 2086. As shown in FIG. 44, the method may include folding drape 2000, such as at least a portion of table drape 2086, along a line H1 toward another portion of table drape 2086. As shown in FIG. 45, the method may include adding one or more labels 2114, such as labels 2114 shown in FIGS. 32-37, such as breakaway label 2116, to drape 2000, such as table drape 2086. As shown in FIG. 46, the method may include accordion folding an internal portion of drape 2000, such as at least a portion of table drape 2086. As shown in FIG. 47, the method may include folding one or more corners of drape 2000, such as corners of a portion of table drape 2086, toward narrower portions of drape 2000, such as a portion of arm drape 2086. As shown in FIG. 48, the method may include tucking in excess material along drape 2000. As shown in FIG. 49, the method may include adding one or more labels 2114, such as labels 2114 shown in FIGS. 32-37, such as table orientation label 2098 and insert hand label 2100, to drape 2000, such as table drape 2086. As shown in FIG. 50, the method may include folding a portion of arm drape 2086 toward drape 2000, such as a portion of table drape 2086, such that hard point 2040 faces toward drape 2000.

Folded drape 2000 may be seen in FIG. 51, and may be compact. Folded drape 2000 may include a thickness of between about 0.5 inch and about three inches, such as between about one inch and about two inches, such as about 1.5 inches. A width of folded drape 2000 may be between about seven inches and about 12 inches, such as between about eight inches and about 10 inches, such as about 8 inches. A length of folded drape 2000 may be between about 10 inches and about 18 inches, such as between about 12 inches and about 16 inches, such as about 14 inches.

Drape 100 (FIG. 1) may be folded such that hard point 140 (FIG. 1) is facing toward drape 100 similar to the folding of drape 2000. In addition, the side of drape 100 that contacts sterile space 10 (FIG. 1) is folded inward to avoid or limit exposure to working space 12 (FIG. 1) when unpackaged. Drape 1000 (FIG. 21) may be folded such that hard point 1040 (FIG. 21) is facing toward drape 1000 similar to the folding of drape 2000. In addition, the side of drape 1000 that contacts sterile space 10 (FIG. 21) is folded inward to avoid or limit exposure to working space 12 (FIG. 21) when unpackaged.

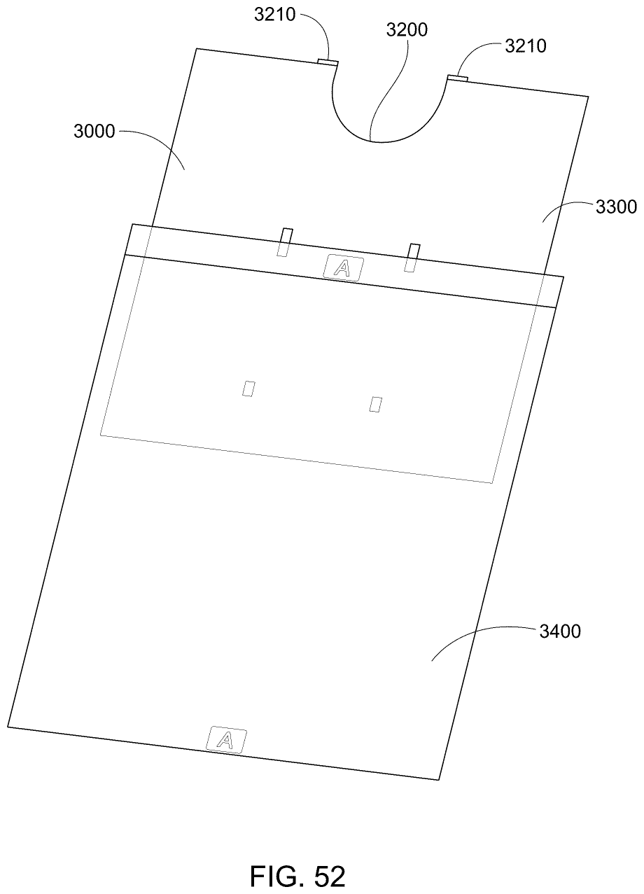

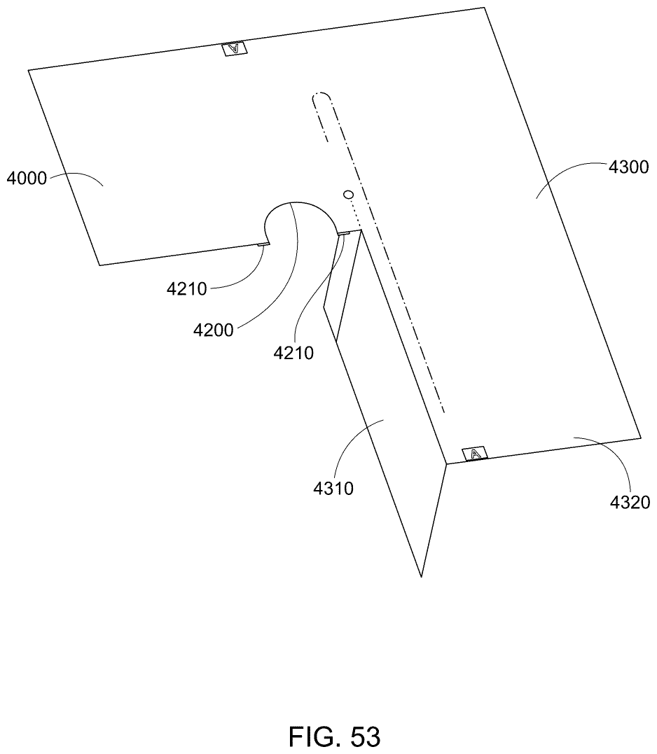

The drapes described above, e.g., drape 100 (FIG. 1), drape 1000 (FIG. 21), and drape 2000 (FIG. 28) may be top drapes. In other words, the drapes may be in contact with sterile space 10 as a last, or final, barrier between working space 12 and sterile space 10. One or more lower drapes may be disposed between the top drapes and working space 12. As shown in FIGS. 52-53, one or more lower drapes may be disposed between a top drape and working space 12, according to exemplary embodiments.

FIG. 52 shows a lower drape 3000. Lower drape 3000 may be disposed under one or more drapes, such as one or more additional lower drapes, one or more top drapes, or one or more additional lower drapes and one or more top drapes. Lower drape 3000 may be disposed under drape 1000 (FIG. 21), for example. Lower drape 3000 may include a leg opening 3200 that receives a leg of a patient, e.g., a right leg or a left leg of a patient. Leg opening 3200 may be u-shaped. Ends of the u-shape may extend down the leg of the patient and may include one or more adhesives 3210 that secure to the patient and keep lower drape 3000 on the patient. Lower drape 3000 may be secured to the patient at leg opening 3200 such that a majority of lower drape 3000 extends away from the leg toward a torso of the patient. Lower drape 3000 may include a fabric portion 3300 that may include leg opening 3200 and one or more adhesive portions 3210. Lower drape 3000 may also include a film 3400. Film 3400 may be attached to fabric portion 3300.

FIG. 53 shows a lower drape 4000. Lower drape 4000 may be disposed under one or more drapes, such as one or more additional lower drapes, one or more top drapes, or one or more additional lower drapes and one or more top drapes. Lower drape 4000 may be disposed under drape 1000 (FIG. 21), for example. Lower drape 4000 may be disposed over drape 3000 (FIG. 52) and under drape 1000, for example. Lower drape 4000 may include a leg opening 4200 that receives a leg of a patient, e.g., a right leg or a left leg of a patient. As shown, leg opening 4200 may receive the right leg of the patient. Leg opening 4200 may be u-shaped. Ends of the u-shape may extend down the leg of the patient and may include one or more adhesives 4210 that secure to the patient and keep lower drape 3000 on the patient. Lower drape 4000 may be secured to the patient at leg opening 3200 such that a majority of lower drape 4000 extends away from the leg toward a torso of the patient. Lower drape 3000 may include a fabric portion 4300 that may include leg opening 3200 and one or more adhesive portions 4210.

As shown, lower drape 4000 is for a right leg of the patient. Accordingly, a right leg 4310 of lower drape 4000 may be detached from a remainder of lower drape 4000. Right leg 4310 being detached may allow for the right leg of the patient to be exposed for a procedure. A left leg 4320 of lower drape 4000 may remain attached to lower drape 4000 to maintain coverage of a left leg of the patient when it is not subject to the procedure. Lower drape 4000 may be configured for a procedure on the left leg of the patient. In this way, leg opening 4200 may receive the left leg of the patient, left leg 4320 of lower drape 4000 may be detached from a remainder of lower drape 4000, and right leg 4310 of lower drape 4000 may remain attached to lower drape 4000.

It should be noted that the use of particular terminology when describing certain features or embodiments of the disclosure should not be taken to imply that the terminology is being re-defined herein to be restricted to include any specific characteristics of the features or embodiments of the disclosure with which that terminology is associated. Terms and phrases used in this application, and variations thereof, especially in the appended claims, unless otherwise expressly stated, should be construed as open-ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least”; the term “such as” should be interpreted as “such as, without limitation”; the term “includes” should be interpreted as “includes but is not limited to”; the term “example” is used to provide exemplary instances of the item in discussion, not an exhaustive or limiting list thereof, and should be interpreted as “example, but without limitation”; adjectives such as “known,” “normal,” “standard,” and terms of similar meaning should not be construed as limiting the item described to a given time period or to an item available as of a given time, but instead should be read to encompass known, normal, or standard technologies that may be available or known now or at any time in the future; and use of terms like “preferably,” “preferred,” “desired,” or “desirable,” and words of similar meaning should not be understood as implying that certain features are critical, essential, or even important to the structure or function of the present disclosure, but instead as merely intended to highlight alternative or additional features that may or cannot be utilized in a particular embodiment.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

In addition, as used herein, the term “coupled” means connected to or joined to or placed into communication with, either directly or through intermediate components. The term “patient” is broadly defined herein to include human patients of all sizes, genders and demographics, as well as animals (e.g., for veterinarian purposes). The terminology includes the words noted above, derivatives thereof and words of similar import.

It will be appreciated by those skilled in the art that changes could be made to the embodiments described above without departing from the broad inventive concept thereof. For example, various mechanical and electrical connection elements and actuators may be used to achieve the disclosed function. It is understood, therefore, that this invention is not limited to the particular embodiments disclosed, but it is intended to cover modifications within the spirit and scope of the present invention as defined by the appended claims.

What is claimed is:

1. A system, comprising:
a medical tool mount disposed in a working space for a surgical procedure and comprising a medical tool mount shape;
a medical tool disposed in a sterile space for the surgical procedure and coupled to the medical tool mount, the medical tool comprising a connector comprising generally opposing sides; and
a drape configured to separate the sterile space from the working space, the drape comprising:
a proximal end,
a distal end,
a drape top surface configured to extend between the proximal end and the distal end of the drape and further configured to define the boundary of the sterile space,
a drape bottom surface opposing the drape top surface, the drape bottom surface configured to extend between the proximal end and the distal end of the drape and further configured to define the boundary of the working space, and
a hard point integral to at least the drape top surface and disposed between the medical tool mount and the medical tool, the hard point comprising:
a hard point top surface configured to contact the sterile space,
a hard point bottom surface configured to contact the working space, and
a flexible portion comprising a flexible material configured to releasably grip the connector of the medical tool on at least the generally opposing sides of the connector, the hard point configured to form a sterile barrier between the medical tool mount disposed in the working space and the medical tool disposed in the sterile space and coupled to the medical tool mount,
wherein the flexible portion is configured to move in conjunction with the medical tool mount as it releasably grips at least the generally opposing sides of the connector of the medical tool, such that the hard point maintains the sterile barrier before and after the flexible portion releasably grips at least the generally opposing sides of the connector.

2. The system of claim 1, the flexible material comprises a flexible material shape, and wherein the medical tool mount comprises a claw having a claw shape, the flexible material shape comprising a corresponding claw shape.

3. The system of claim 1, wherein the hard point further comprises a recessed pocket configured to receive at least a portion of the connector of the medical tool, the connector comprising a head portion comprising a first diameter and a shaft comprising a second diameter, the first diameter larger than the second diameter.

4. The system of claim 3, wherein the recessed pocket comprises a bottom shape, and
wherein the head portion comprises a head portion shape, the bottom shape corresponding to the head portion shape.

5. The system of claim 1, wherein the medical tool is a foot mount.

6. The system of claim 1, wherein the flexible portion of the hard point comprises a first end and a second end configured to move generally toward the first end to releasably grip the connector of the medical tool on at least the generally opposing sides of the connector.

7. The system of claim 6, wherein the first end is configured to move toward the second end.

8. The system of claim 1, wherein the drape further comprises a drape material, and
wherein the hard point is more rigid than the drape material.

9. The system of claim 1, wherein the hard point is less rigid than the connector.

10. A system, comprising:
a medical tool mount disposed in a working space for a surgical procedure and comprising a medical tool mount shape;
a medical tool disposed in a sterile space for the surgical procedure and coupled to the medical tool mount, the medical tool comprising a connector comprising generally opposing sides; and
a drape configured to separate the sterile space from the working space, the drape comprising:
a proximal end,
a distal end,
a drape top surface configured to extend between the proximal end and the distal end of the drape and further configured to define the boundary of the sterile space,
a drape bottom surface opposing the drape top surface, the drape bottom surface configured to extend between the proximal end and the distal end of the drape and further configured to define the boundary of the working space, and
a hard point integral to at least the drape top side and disposed between the medical tool mount and the medical tool, the hard point comprising:
a hard point top surface configured to contact the sterile space,
a hard point bottom surface configured to contact the working space,
a recessed pocket configured to receive the head of the connector,
a frame surrounding the recessed pocket and configured to receive the shaft of the connector, and
a flexible portion comprising a flexible material configured to releasably grip the connector of the medical tool on at least the generally opposing sides of the connector, the hard point configured to form a sterile barrier between the medical tool mount disposed in the working space and the medical tool disposed in the sterile space and coupled to the medical tool mount,
wherein the flexible portion is configured to move in conjunction with the medical tool mount as it releasably grips at least the generally opposing sides of the connector of the medical tool, such that the hard point maintains the sterile space and the working space before and after the flexible portion releasably grips at least the generally opposing sides of the connector, and
wherein the recessed pocket and the frame comprise the flexible material such that the recessed pocket and the frame are configured to cinch at least the generally opposing sides of the connector.

11. The system of claim 10, wherein the hard point further comprises:
a rear surface,
a recessed cavity formed in the recessed pocket, and an outer frame surrounding the recessed pocket and the frame, and
wherein the frame comprises:
transverse walls extending generally transversely from the rear surface,
a front surface generally parallel to the rear surface,
a frame opening formed in the front surface and comprising a v-shaped edge, and
a frame cavity bounded by the rear surface, the transverse walls, and the front surface,
wherein the recessed cavity extends below the frame cavity,
wherein the outer frame is circular and oblique relative to the front surface of the frame,
wherein the recessed pocket extends outwardly from and below the outer frame,
wherein the connector further comprises a head portion comprising a first diameter and a shaft comprising a second diameter, the first diameter larger than the second diameter,
wherein the recessed pocket comprises a bottom shape, the recessed cavity comprises a recessed cavity shape, and the head portion comprises a head portion shape, the recessed cavity shape corresponding to the bottom shape and the head portion shape, the recessed cavity shape, the bottom shape, and the head portion shape being octagonal, and
wherein the frame opening has a frame diameter corresponding to the second diameter such that the frame opening is configured to receive the shaft but not the head portion comprising the first diameter, such that the head portion may be retained in the recessed cavity by the frame when the hard point is coupled to the medical tool mount.

12. The system of claim 10, wherein the frame comprises a frame opening comprising a v-shaped edge.

13. The system of claim 12, wherein the frame opening comprises a frame opening shape, and wherein the shaft comprises a shaft shape, the frame opening shape corresponding to the shaft shape.

14. The system of claim 10, wherein the hard point further comprises an outer frame surrounding the recessed pocket and the frame.

15. The system of claim 14, wherein the frame comprises a front surface, and wherein the outer frame is oblique relative to the front surface of the frame.

16. A system, comprising:
a medical tool mount disposed in a working space for a surgical procedure and comprising a medical tool mount shape;
a medical tool disposed in a sterile space for the surgical procedure and coupled to the medical tool mount, the medical tool comprising a connector comprising generally opposing sides; and
a drape configured to separate the sterile space from the working space, the drape comprising:
a proximal end,
a distal end,
a drape top surface configured to extend between the proximal end and the distal end of the drape and further configured to define the boundary of the sterile space,
a drape bottom surface opposing the drape top surface, the drape bottom surface configured to extend between the proximal end and the distal end of the drape and further configured to define the boundary of the working space, and
a hard point integral to at least the drape top side and disposed between the medical tool mount and the medical tool, the hard point comprising:
a hard point top surface configured to contact the sterile space,
a hard point bottom surface configured to contact the working space,
a first end,
a second end, and
a flexible portion comprising a flexible material configured to releasably grip the connector of the medical tool on at least the generally opposing sides of the connector, the hard point configured to form a sterile barrier between the medical tool mount disposed in the working space and the medical tool disposed in the sterile space and coupled to the medical tool mount,
wherein the flexible portion is configured to move in conjunction with the medical tool mount as it releasably grips at least the generally opposing sides of the connector of the medical tool, such that the hard point maintains the sterile space and the working space before and after the flexible portion releasably grips at least the generally opposing sides of the connector, and
wherein the second end is configured to move toward the first end to couple the medical tool to the medical tool mount.

17. The system of claim 16, wherein the hard point further comprises:
a channel extending between the first end and the second end, the channel configured to receive at least a portion of the connector, and
a bottom surface of the channel, the first end extending above the second end relative to the bottom surface of the channel,
wherein the bottom surface of the channel is oblique relative to the hard point bottom surface, and
wherein the flexible material comprises a flexible material shape, and wherein the medical tool mount comprises a claw having a claw shape, the flexible material shape corresponding to the claw shape such that the second end is configured to move toward the first end to couple the medical tool to the medical tool mount.

18. The system of claim 16, wherein the first end is configured to move toward the second end to couple the medical tool to the medical tool mount.

19. The system of claim 16, wherein the flexible material comprises a flexible material shape, and wherein the medical tool mount comprises a claw having a claw shape, the flexible material shape corresponding to the claw shape.

20. The system of claim 16, wherein the hard point further comprises a receiving opening configured to receive at least a portion of the connector of the medical tool.
